(19) United States
(12) Patent Application Publication
(10) Pub. No.: US 2022/0325242 A1
(43) Pub. Date: Oct. 13, 2022

(54) IDH2 INHIBITION FOR PRODUCING T-CELLS AND B-CELLS WITH A MEMORY PHENOTYPE

(71) Applicant: Universite de Lausanne, Lausanne (CH)

(72) Inventors: Pedro ROMERO, Epalinges (CH); Mathias WENES, Chexbres (CH)

(21) Appl. No.: 17/637,270

(22) PCT Filed: Aug. 21, 2020
(86) PCT No.: PCT/EP2020/073551

§ 371 Date: Feb. 22, 2022

(30) Foreign Application Priority Data
Aug. 21, 2019 (EP) 19192887.8

(51) Int. Cl. C12N 5/0783; A61K 35/17

(52) U.S. Cl. CPC C12N 5/0636; A61K 35/17; C12N 2501/999

(57) ABSTRACT

The present invention relates to an in vitro cell culture method comprising a step of contacting T-cells with an IDH2 inhibitor, and further to a cell population comprising T-cells with a memory phenotype obtained by said method, preferably, wherein the T-cells are human cells. The present invention also relates to a method for generating and/or maintaining T-cells and/or B-cells with a memory phenotype comprising the steps of culturing T-cells and/or B-cells in vitro and adding an IDH2 inhibitor to the culture. The invention furthermore relates to a population of T-cells and/or B-cells obtained by the methods of the invention. Also provided are immunotherapies using the cells of the invention. Furthermore, provided is an IDH2 inhibitor for use in immunotherapy.

The present invention relates to an in vitro cell culture method comprising a step of contacting T-cells with an IDH2 inhibitor, and further to a cell population comprising T-cells with a memory phenotype obtained by said method, preferably, wherein the T-cells are human cells. The present invention also relates to a method for generating and/or maintaining T-cells and/or B-cells with a memory phenotype comprising the steps of culturing T-cells and/or B-cells in vitro and adding an IDH2 inhibitor to the culture. The invention furthermore relates to a population of T-cells and/or B-cells obtained by the methods of the invention. Also provided are immunotherapies using the cells of the invention. Furthermore, provided is an IDH2 inhibitor for use in immunotherapy.

Immunotherapy aims to activate or suppress the immune system for treating a variety of diseases such as cancer, autoimmune diseases and chronic viral infections. Traditionally, immunomodulatory cytokines, monoclonal antibodies and inhibitors or agonists of signaling molecules are used to modulate and direct the activity of the immune system. Although therapeutic effects have been achieved using those compounds, they do not allow to exploit the full potential of immunotherapy. This is inter alia due to the limited half-life of the compounds in a patient and the merely indirect mode of action via stimulation of endogenous immune cells. Thus, recently, cell-based immunotherapies have been developed wherein immune cells, such as T-cells or B-cells, are transferred into a patient. One application is the (re-)establishment of a functional immune response in patients; Winkler et al., Blood 2016;128:502. Most typically, however, the immune cells, in particular cytotoxic (CD8+) T-cells, have been selected or engineered/redirected to eliminate certain cells, in particular cancer cells, expressing specific antigens; e.g. Gattinoni, Semin Immunol. 2016 Feb;28(1):1-2; Hammerl et al., Trends Immunol. 2018 Nov;39(11):921-936; Pettitt et al., Mol Ther. 2018 Feb 7;26(2):342-353. Additionally, T-cell therapies are being developed to target certain immune cells causing autoimmune diseases or cells infected by viruses; see e.g. Maldini et al.; Nat Rev Immunol. 2018 Oct;18(10):605-616. In addition to exploiting the cytotoxic activity of CD8+ T-cells, T-cell therapy, in particular based on CD4+ T-cells, can be used to modulate the signaling environment at the target site and activate or inhibit other immune cells. Helper (CD4+) T-cells have been applied, for example, to enhance the immune response of CD8+ T-cells, see e.g. Li et al., Clin Transl Immunology. 2016 Aug 12;5(8):e95. A subset of CD4+ T-cells, called “regulatory T-cells”, has been also used to suppress the immune system for the treatment of autoimmune diseases; Romano et al., Front Immunol. 2019 Jan 31;10:43.

Cancer cells naturally present certain antigens on their surface which can be recognized by naïve CD8+ T-cells and which contribute to their development into cancer-specific cytotoxic T-cells. Cancer-antigens are also taken up by antigen-presenting cells (APCs) which leads to activation of CD4+ T-cells which provide further stimuli for the activation of CD8+ T-cells. Thus, the immune system can intrinsically attack cancer cells, but it usually fails to fully eliminate the cancer. Cancer immunotherapies aim to elicit a stronger immune response against cancer in the hope to significantly expand the life-span of the patient or even achieve full remission. In one modality called adoptive cell transfer therapy (ACT), autologous cells, preferably T-cells, are obtained from the patient, expanded in vitro and transferred back into the patient. For example, cancer-specific T-cells and/or B-cells are extracted from tumor biopsies of the patient (tumor infiltrating lymphocytes, TILs). In vitro culture then allows, in principle, further selecting, manipulating and activating of the T-cells to produce highly effective and specific T-cells. As alternative to wild-type cancer-specific cells, naïve T-cells can be obtained and manipulated by genetic engineering to express specific T-cell receptors (TCR) to recognize tumor-associated antigens (TAA) or tumor-specific antigens (TSA/neoantigens). Another way of redirecting T-cells towards cancer cells is by introducing a chimeric antigen receptor (CAR) which recognizes a TAA or TSA and promotes activation of the T-cells.

However, both the magnitude of tumor responses and the fraction of patients benefitting from those novel therapeutic approaches remain limited. T-cells prepared for ACT are generally terminally differentiated, resulting in inefficient engraftment and cancer recurrence; Gattinoni et al., Nat Rev Cancer. 2012 Oct;12(10):671-84.

Certain subsets of memory T-cells, however, are long-lived while maintaining the capacity to differentiate into effector T-cells, hence showing properties of stem cells. T-cells with a memory phenotype are thought to elicit a more durable and effective anti-cancer response upon transfer into a subject; Klebanoff et al.; Clin Cancer Res. 2011 Aug 15;17(16):5343-52 and Klebanoff et al,; JCI Insight. 2017;2(23):e95103. Several markers have been established to distinguish T-cells or B-cells based on their differentiation status. Expression of CD62L or CCR7 at the cell surface, for example, marks (stem) central memory T-cells; Gattinoni et al., Nat Rev Cancer. 2012 Oct;12(10):671-84. Furthermore, the metabolic profile shifts markedly during T-cell or B-cell differentiation. Effector T-cells, for example, have a strong anabolic metabolism with increased aerobic glycolysis, whereas memory T-cells rely on a mitochondrial metabolism and fatty acid oxidation; Kishton et al., Cell Metab. 2017 Jul 5;26(1):94-109. There have also been some attempts to control the differentiation process during the in vitro expansion phase to enhance formation of T-cells with a memory phenotype, for example by inhibition of AKT, PIK3 or mTOR or by application of 2-deoxyglucose or exogenous arginine to reduce glycolysis; Sukumar et al., J Clin Invest. 2013 Oct;123(10):4479-88; Crompton et al., Cancer Res. 2015 Jan 15;75(2):296-305; Klebanoff et al., JCI Insight. 2017 Dec 7;2(23); Geiger et al., Cell. 2016 Oct 20;167(3):829-842.e13; Scholz et al., EBioMedicine. 2016 Jan 16;4:50-61; Klebanoff et al,; JCI Insight. 2017;2(23):e95103. Those protocols, however, do not provide an optimal yield, negatively interfere with the activation of T-cells, and/or, as found in the context of the present invention for AKT inhibition, negatively interfere with the reactivation of memory-like T-cells. Furthermore, it may depend on the therapeutic purpose, if T- or B-cells with a memory phenotype should be, for example, highly proliferative or rather quiescent when transferred into a patient. The exact memory phenotype of T- or B-cells obtained after in vitro expansion, however, likely depends on the culture protocol.

Thus, there is a need for improved methods to obtain T-cells and/or B-cells, in particular T-cells, with a memory phenotype. There is also a need to expand the repertoire of compounds which can promote a memory phenotype during in vitro expansion of T- or B-cells for the development of new immunotherapies.

Accordingly, the invention relates to an in vitro cell culture method comprising a step of contacting T-cells with an IDH2 inhibitor. In particular, T-cells with a memory phenotype are generated and/or maintained by said method. Preferably, an increased proportion of T-cells with a memory phenotype is thereby generated, i.e. compared to an in vitro cell culture method which does not employ an IDH2 inhibitor but is preferably otherwise identical (e.g. a control cell culture method).

Thus, the invention also relates to a method for generating and/or maintaining T-cells and/or B-cells with a memory phenotype comprising the steps of culturing T-cells and/or B-cells in vitro and adding an IDH2 inhibitor to the culture.

In particular herein, and in the context of the invention, when reference is made to T-cells and/or B-cells, said cells are preferably T-cells. Preferably herein, the T-cells are or comprise CD8+ T-cells. Furthermore, the terms “CD8+ T-cells” and “cytotoxic T-cells” are used interchangeably herein.

The invention is, at least partly, based on the surprising discovery that T-cells, in particular CD8+ T-cells, with a memory phenotype can be obtained from in vitro culture, when an IDH2 (isocitrate dehydrogenase (NADP(+)) 2, mitochondrial) inhibitor is added to the culture, or in other words, when the T-cells are contacted with an IDH2 inhibitor during culture, as demonstrated in the appended Examples. In particular, the obtained T-cells are enriched for T-cells with a memory phenotype. It is further a surprising discovery that said T-cells can be efficiently activated and obtained with high efficiency, despite having a memory phenotype. In addition, it was surprisingly found by the inventors that CD8+ T-cells exist at higher numbers and comprise more cells with a memory phenotype in vivo upon adoptive cell transfer when they have been cultured in the presence of an IDH2 inhibitor in vitro. It was also surprisingly found that cancer-specific adoptively transferred CD8+ T-cells have an enhanced anti-cancer activity in vivo when they have been cultured in the presence of an IDH2 inhibitor in vitro. The present invention provides thus more effective cancer-specific CD8+ T-cells, which may be used to improve commonly practiced anti-cancer T-cell immunotherapies. The invention is, however, not limited to CD8+ T-cells, but an IDH2 inhibitor may be also used to obtain other T-cells, e.g. CD4+ T-cells, or B-cells with a memory phenotype. Furthermore, the inventors have found that contacting T-cells during culture with an IDH2 promotes the generation of T-cells with a memory phenotype with both, mouse and human cells. In particular, since the key findings with mouse T-cells could be reproduced with human cells, i.e. mononuclear cells from umbilical cord blood (CBMCs), the culture methods and cell populations of the invention may be highly useful for T-cell based immunotherapies for human patients, e.g. cancer patients. Moreover, it was found that the human CD8+ T-cells with a memory phenotype that were produced by culturing human T-cells in the presence of an IDH2 inhibitor were more effectively restimulated than human CD8+ T-cells with a memory phenotype that were produced by culturing in the absence of an IDH2 inhibitor but in the presence of an AKT inhibitor. In fact, AKT inhibition has surprisingly even a negative effect. Thus, the data illustrated in the appended Examples indicate that one of the main characteristics of memory T-cells, namely their ability to react with an increased amplitude to a reencounter of the antigen, is enhanced when the T-cells have been contacted with an IDH2 during culture, whereas this ability may be decreased when the memory-like T-cells are generated differently, i.e. by contacting with an AKT inhibitor during culture (see Klebanoff et al,; JCI Insight. 2017;2(23):e95103). Thus, the present invention further provides more effective T-cells and/or B-cells, i.e. more effective memory-like T-cells, which may be used to develop and/or improve further immunotherapies. The use of T-cells and/or B-cells of the invention which are more effective may thus lead to improved immunotherapies, in particular anti-cancer T-cell immunotherapies, wherein less cells have to be transferred into a patient. Furthermore, said T-cells or antibodies produced by said B-cells may be targeted to non-cancerous cells such as immune cells promoting an autoimmune disease or cells infected by a virus. The target cells may be killed, and/or their activity or cell state may be altered, for example, by modulation of their signaling environment due to their proximity to transferred T-cells, and/or by bound antibodies produced by transferred B-cells. T-cells or B-cells with a memory phenotype obtained upon culture with an IDH2 inhibitor may generally provide a more sustained therapeutic effect compared to rather short-lived, or even senescent, effector T-cells and/or B-cells that are commonly used for cell immunotherapies.

Without being bound by theory, it is believed that IDH2 inhibition leads to the depletion and/or accumulation of metabolites that influence the epigenetic state of the T-cells and/or B-cells, which promotes the transcription of memory-associated genes. In fact, as illustrated in the appended Examples, IDH2 inhibition leads to a more open chromatin configuration, inter alia near memory-phenotype-associated genes, which is characterized by an increased trimethylation of lysine residue 4 and increased acetylation of lysine residue 27 of histone H3, and more accessible regions in the DNA as determined by ATAC-seq. These data further confirm that transient inhibition of IDH2 during the initial activation phase of T-cells in the culture is sufficient, and has a long-lasting effect, and i.e. promotes the generation and/or maintenance of T-cells with a memory phenotype. However, as illustrated in the appended Examples, the promotion of a memory phenotype may be also achieved or even further enhanced when the IDH2 is present throughout the entire culture period, e.g. for the first week of culture time for murine T-cells, or for the first 10 or 11 days for human T-cells, respectively. Yet, it is believed that further in vitro expansion, as well as adoptive transfer to tumor bearing hosts, no longer requires the contact with the IDH2 inhibitor. Thus, once the epigenetic state is modified, the reshaped chromatin configuration is stable and is transmitted to the progeny of thus modified T-cells and/or B-cells.

Thus, IDH2 inhibition may promote differentiation of naïve T-cells and/or B-cells into T-cells and/or B-cells with a memory phenotype and/or promote the maintenance of T-cells and/or B-cells with a memory phenotype during in vitro culture.

In a preferred embodiment, the T-cells and/or B-cells are activated before and/or during culture, preferably during culture, in particular while the cells are contacted with the IDH2 inhibitor, and preferably from beginning of the culture. Activated T-cells and/or B-cells with a memory phenotype may be preferable for adoptive cell transfer therapies to rapidly generate a large amount of antigen-specific effector cells in vivo, but without rapid exhaustion of the transferred cell pool. In vitro cell culture comprising an IDH2 inhibitor allows activation of naïve T-cells and/or B-cells and/or allows maintenance of activated T-cells and/or B-cells during in vitro culture. Moreover, as shown in the appended Examples, the T-cells, i.e. the human T-cells, generated according to the method of the invention can be effectively reactivated, and respond to a restimulation cue with an increased production of Interferon-gamma (IFNγ) compared to T-cells that were cultured in the absence of an IDH2 inhibitor, or with an AKT inhibitor instead. This suggests that the T-cells with a memory phenotype provided herein have superior properties, which may be highly advantageous for their use in immunotherapy.

In certain embodiments, IL-2, IL-7 and/or one or more antigenic peptide(s) are added to the culture. In particular, IL-2 is added during activation and the contacting with the IDH2 inhibitor provided herein. IL-2, IL-7 and/or one or more antigenic peptide(s) further promote the generation and/or maintenance of activated T-cells and/or B-cells with a memory phenotype.

Accordingly, the invention further relates to a cell population comprising T-cells with a memory phenotype obtained by inventive method provided herein, preferably wherein the T-cells are human cells. Said cell population is also called a population of T-cells herein, because it is preferred that the cell population consists predominantly or exclusively of T-cells, for example, wherein at least 90%, 95%, 98% or 99% of the cells in the population are T-cells. Thus, said cell population may be a population of T-cells obtained by the method of the invention which comprises a higher proportion of T-cells with a memory phenotype and/or shows in average a more pronounced memory phenotype compared to a population of T-cells obtained in parallel by the same method except that no IDH2 inhibitor is added to the culture (DMSO control). Moreover, it is preferred that at least 30%, 40%, 50%, 60%, 70%, 80%, or 90%, or at least 95%, 97%, 98% or 99%, preferably at least 70%, of the cells in the inventive cell population provided herein are T-cells with a memory phenotype, in particular wherein said T-cells with a memory phenotype express CD62L.

The cell population provided herein may be used in immunotherapy as described herein, in particular wherein the cell population or the T-cells comprised in said cell population is/are administered to a patient.

Furthermore, the invention relates to a population of T-cells and/or B-cells obtained by the method of the invention which comprises a higher proportion of T-cells and/or B-cells with a memory phenotype and/or shows in average a more pronounced memory phenotype compared to a population of T-cells and/or B-cells obtained in parallel by the same method except that no IDH2 inhibitor is added to the culture (DMSO control). In one embodiment, the population of cells are mouse cells, and the proportion of cytotoxic T-cells showing surface expression of CD62L after about 7 days of culture is about 60 to 75%.

In one embodiment, the population of cells are mouse cells, and the mean fluorescence intensity of CD62L in cytotoxic T-cells therein is about 1.5-fold to 2-fold increased compared to a DMSO control.

Accordingly, the invention further relates to an IDH2 inhibitor for use in immunotherapy. In particular, the immunotherapy may comprise administering T-cells to a patient, wherein the T-cells have been contacted with the IDH2 inhibitor during the in vitro culture according to the inventive method provided herein. In particular, said T-cells have acquired a memory phenotype during said in vitro culture.

Accordingly, the invention further relates to a population of T-cells and/or B-cells obtained by the method of the invention, i.e. a cell population comprising T-cells with a memory phenotype as provided herein, for use in immunotherapy.

Accordingly, the invention further relates to an immunotherapy comprising administering an IDH2 inhibitor and/or a population of T-cells and/or B-cells contacted with an IDH2 inhibitor to a patient.

In certain embodiments, the immunotherapy comprises T-cells and/or B-cells which acquire or have acquired a memory phenotype within a subject, in vitro and/or ex vivo.

The application of an IDH2 inhibitor to promote the generation and/or maintenance of T-cells and/or B-cells with a memory phenotype is not limited to a specific time between the isolation of primary cells and the end of the cell therapy or a specific location of the cells. However, it is preferred that the T-cells are contacted with the IDH2 inhibitor at least during activation, preferably from the beginning of the culture and/or activation, for example for the first 3 or 4 days. Furthermore, the T-cells may be contacted with the IDH2 inhibitor during the entire culture period. However, it is also possible to wash the IDH2 inhibitor away after the initial activation phase (priming phase), and continue the culture without an IDH2 inhibitor, e.g. with a medium comprising IL-2 and IL-7.

In particular, the activation phase, which may also be called “the priming phase”, may take place from day 0 to day 3 or 4, i.e. for the first three or four days of the culture. During that phase, the foundations for effector vs memory differentiation are made. Towards the end of the culture, the T cells are fully differentiated and matured into memory T cells, for example until day 7 for mouse cells, or day 10 to 11 for human T-cells. As described herein, the IDH2 inhibitor is preferably present in the activation phase but may be still present during the subsequent maturation phase. At the end of the culture, the T-cells with a memory phenotype may be restimulated to determine their reactivation capacity (recall potential), as described herein.

An IDH2 inhibitor may be administered to T-cells and/or B-cells within a subject (in vivo), in vitro and/or ex vivo. The T-cells and/or B-cells may be in a culture dish/flask or be within a subject, for example in the blood, lymph or tissue, for example a lymphoid organ or a tumor, when contacted with an IDH2 inhibitor. Preferably, the T-cells are contacted with the IDH2 inhibitor in vitro during culture.

In a preferred embodiment, the immunotherapy is a T-cell therapy. Preferably, the T-cell therapy comprises CD8+ (cytotoxic) T-cells. Cytotoxic T-cells with a memory phenotype obtained upon contact with an IDH2 inhibitor may persist for a prolonged period of time within a subject, for example, upon adoptive cell transfer. They may also produce a larger number of cytotoxic effector T-cells for a longer time which may cause efficient lysis of the target cells. A sustained cytotoxic activity of transferred CD8+ T-cells with a memory phenotype may lead to a durable depletion, and in some cases even permanent elimination, of the target cells, for example, cancer cells.

In certain embodiments, the immunotherapy is a therapy to treat cancer, a chronic viral infection or an autoimmune disease.

Cytotoxic T-cells with a memory phenotype are particularly suitable to eliminate cells, i.e. mediate the elimination of cells, for example cancer cells or cells infected by a virus. Cancer cells or cells infected by virus are preferred targets, at least partly because they may express specific neoantigens or virus-derived antigens which may reduce the risk of non-specifically targeting healthy cells. Certain subsets of CD4+ T-cells (helper T-cells) with a memory phenotype may also contribute to the elimination of target cells, either directly by generating cells with a cell-lytic activity or indirectly by stimulating/activating CD8+ T-cells. CD4+ regulatory T-cells with a memory phenotype, in contrast, may promote an immunosuppressive environment, for example in certain tissues and/or in proximity of certain T-cells or B-cells. Such an immunosuppressive activity may be particularly desired for the treatment of autoimmune diseases.

In a preferred embodiment, the immunotherapy is a therapy to treat cancer. T-cells and/or B-cells with a memory phenotype may be also effective in the treatment of aggressive and/or late-stage cancer.

In a preferred embodiment, the cancer is resistant to chemotherapy, targeted therapy and/or antibody-mediated immunotherapy and/or comprises metastases.

In particular herein, e.g. in certain embodiments, the immunotherapy may comprise transfer of in vitro or ex vivo cultured T-cells and/or B-cells into a subject. In particular, T-cells and/or B-cells are obtained from a subject, preferably the patient (autologous cells), cultured in vitro (ex vivo) and adoptively transferred into the patient.

In a preferred embodiment of the invention, the cells are T-cells. In a very preferred embodiment, the T-cells comprise cytotoxic T-cells (CD8+ T-cells).

In certain embodiments, the memory phenotype comprises higher expression of CD62L, TCF1, CD127, CCR7, CD27 and/or CD28, lower expression of KLRG1, and/or an increased basal oxygen consumption, maximal respiratory capacity and/or spare respiratory capacity compared to a control. In particular, in the context of the present invention, the control refers to a control treatment of T-cells and/or B-cells which does not comprise an IDH2 inhibitor and is preferably a solvent control, for example comprising DMSO. Preferably, the control treatment is performed in parallel with a comparable population of cells and, where necessary, comparable subjects, and all steps are identical except that the respective solvent is used instead of the IDH2 inhibitor. When the protein is expressed at the cell surface, it is preferably detected at the cell surface. An increased basal oxygen consumption, maximal respiratory capacity and/or spare respiratory capacity is indicative of a mitochondrial metabolism, at least partially through fatty acid oxidation which is associated, as known in the art, with a memory phenotype rather than an effector phenotype (Zhang (2018) Trends Mol Med 24(1):30-48) Preferably, the memory phenotype comprises higher expression of CD62L, preferably at the cell surface.

In certain embodiments, the T-cells and/or B-cells comprise T-cells and/or B-cells derived, i.e differentiated, from tumor-infiltrating lymphocytes (TILs). In particular, the TILs are isolated from a tumor of the same patient which receives the TILs, i.e. the in vitro expanded TILs, upon IDH2 treatment during in vitro culture. Preferably, the TILs are T-cells. TILs are enriched for T-cells specific for the cancer of a patient and therefore a suitable basis for cancer T-cell therapy, i.e. a suitable starting cell population for the inventive culture method provided herein. As known in the art, however, TILs comprise mainly, but not necessarily only, effector and/or senescent cells. IDH2 treatment may thus be useful for enriching TILs with a memory phenotype.

In certain embodiments, the T-cells comprise a heterologous antigen receptor, preferably a T-cell receptor (TCR) or a chimeric antigen receptor (CAR). There are methods known in the art, to identify and select a TCR or generate a CAR which is specific for a certain antigen, see e.g. Weber (2020), Cell 181(1):46-62, and June (2018), N Engl J Med 379(1):64-73. Establishing antigen-specificity by genetic engineering such that the T-cells express the desired TCR or CAR may increase the repertoire of suitable T-cells for in vitro culture and subsequent adoptive cell transfer. In particular, naïve T-cells (which are not yet specific for antigen) can be isolated from a patient or MHC/HLA matched subject, genetically manipulated to express a certain TCR or CAR, activated and differentiated into T-cells with a memory phenotype.

In particular herein, in a preferred embodiment of the invention, the IDH2 inhibitor comprises one or more small molecule(s). In a very preferred embodiment, the small molecule(s) is/are AG221 and/or AGI6780. In other words, most preferably, the IDH2 inhibitor according to the invention comprises at least AG221 and/or AGI6780, in particular wherein AG221 is 2-methyl-1-[[6-(trifluoromethyl)pyridin-2-yl]-6-[[2-(trifluoromethyl)pyridin-4-yl]amino]-1,3,5-triazin-2-yl]amino]propan-2-ol, and AGI6780 is 1-[5-(cyclopropylsulfamoyl)-2-thiophen-3-ylphenyl]-3-[3-(trifluoromethyl)phenyl]urea, or a (or another) pharmaceutically acceptable salt thereof.

In one embodiment, the immunotherapy comprises administering the IDH2 inhibitor to a subject. Without being bound by theory, the IDH2 inhibitor may increase the frequency of T-cells and/or B-cells within a subject when administered to said subject, thus boosting and/or prolonging an immune response and/or extending the protective effect of vaccination. In some embodiments, the IDH2 inhibitor is administered to a subject for treating a different disease than cancer. As described above, the invention relates to an in vitro cell culture method comprising a step of contacting T-cells with an IDH2 inhibitor. In particular, the IDH2 inhibitor is comprised in the medium used for culturing the T-cells. Evidently, when the IDH2 inhibitor is added to an in vitro T-cell culture, i.e. to the culture medium used for culturing the T-cells, said T-cells are contacted with the IDH2 inhibitor. Thus, the T-cells are cultured in the presence of an IDH2 inhibitor according to the inventive method provided herein. In some embodiments, the IDH2 inhibitor is added to the culture medium before the T-cells are cultured with said medium. However, the IDH2 inhibitor may be also added to the culture after the T-cell culture is initiated, e.g. after or during the cells are seeded or incubated, but ideally shortly after culture start. It is preferred that the IDH2 inhibitor is present in the culture medium from the beginning of the culture. Moreover, preferably, the T-cells are at least contacted with the IDH2 inhibitor while being activated, although it is not strictly necessary that the IDH2 inhibitor is present during the entire activation phase.

Hence, the IDH2 inhibitor is used for culturing T-cells, as described herein, in particular for generating and/or maintaining T-cells with a memory phenotype.

Thus, the invention also relates to a method for generating and/or maintaining T-cells and/or B-cells with a memory phenotype comprising the steps of culturing T-cells and/or B-cells in vitro and adding an IDH2 inhibitor to the culture.

Accordingly, the invention further relates to an in vitro cell culture comprising T-cells and an IDH2 inhibitor. Said in vitro cell culture may comprise a culture vessel (e.g. a dish, a well plate, a bioreactor, a flask, or a bottle), a culture medium comprising an IDH2 inhibitor as provided herein, and T-cells. The culture may be static or dynamic (e.g. agitated by means of rotation or stirring).

In a preferred embodiment, the method of the invention further comprises a step of obtaining T-cells and/or B-cells with a memory phenotype from the culture. Thus, the inventive methods provided herein may comprise a further step of obtaining the cells, in particular the T-cells, from the culture, thereby producing a cell population comprising T-cells with a memory phenotype, as described herein.

Preferably herein, the T-cells are human cells, for example, human umbilical cord blood (CB) cells, i.e. mononuclear cells from umbilical cord blood (CBMCs), or peripheral blood mononuclear cells (PBMCs).

Mononuclear cells from blood such as cord blood mononuclear cells (CBMCs) or peripheral blood mononuclear cells (PBMCs) refer to cells from the blood which can, e.g., be easily isolated by Ficoll gradient purification, and which may contain mainly lymphocytes (e.g. T-cells), but also monocytes and dendritic cells. As described herein, the other cells than T-cells among the mononuclear cells (e.g. dendritic cells) may function as antigen-presenting cells and thus positively contribute to the T-cell activation and/or generation of the memory phenotype according to the invention. However, these other cells are not strictly required, e.g., because T-cell activation can be also performed by other means, as described herein, such as anti-CD3 and anti-CD28 antibodies. The terms “cord blood” and “umbilical cord blood” are used interchangeably herein, and even without specification of the term “human”, they refer herein, in particular, to human cord blood. Peripheral blood, as used herein, refers, in particular, to venous blood, i.e. from humans, that may be taken by venipuncture.

Thus, the invention further relates to a cell population comprising human T-cells with a memory phenotype obtained by the method of the invention.

In a preferred embodiment, the T-cells and/or B-cells are activated before and/or during culture, preferably while being cultured.

The term “memory phenotype”, as used herein, is defined as a cell state which resembles a memory T-cell and/or memory B-cell at least in some aspects. The terms “memory-like T-cell” or “memory-like B-cell” also refer to the term “memory phenotype”. An important feature associated with a memory phenotype is the longevity of the cell. Longevity means that the cell or a progenitor survives long enough, e.g. without dividing or slowly dividing, in a subject to be able to elicit a therapeutic effect. In particular, a cell with a memory phenotype has stem cell-like properties. The longevity is preferably due to self-renewal which comprises proliferation. Self-renewal, as used herein, is not meant in a strict sense, but also includes the capacity to maintain a largely similar, although not necessarily identical, phenotype for a therapeutically relevant period of time. The self-renewal can be maintained for the entire life-time or even beyond, but it is sufficient, as used herein, if it is maintained long enough for the therapeutic purpose. A therapeutically relevant period of time means that the transferred cells or their progeny persist long enough in a subject to have a therapeutic effect. While a T-cell and/or B-cell with a memory phenotype is living, and preferably proliferating, it typically maintains the capacity to differentiate into effector cells. The capacity to generate an effector T-cell or B-cell is thus another important feature associated with a memory phenotype. A T-cell and/or B-cell with a memory phenotype thus can produce a higher number of therapeutically active effector cells than effector cells themselves which rather tend to senesce and die early. Important functional features associated with a memory phenotype can be also measured relative to other cell populations. For example, longevity, self-renewal and/or the capacity to differentiate into effector cells may be compared to effector cells, terminally differentiated cells and/or senescent cells.

Another important feature associated with the memory phenotype is the ability of the cells to react with an increased amplitude of (re)activation to a reencounter of the antigen, as is observed, e.g., with memory T-cells.

Memory T-cells are poised to respond to a reencounter of the antigen with a kinetics that is much faster than the primary response from a naive T-cell. Furthermore, memory cells are arrested in G1 of the cell cycle while naïve cells are at G0. This allows for a rapid cell division, as they simply proceed through the cell cycle once recalled by a second encounter with antigen. Likewise, their effector genes are poised for rapid transcriptional activation which leads to a swift cytokine response and generation of a fresh cohort of effector T-cells. In addition, a small proportion of memory cells renew and continue to cycle slowly as a way to preserve the pool of memory cells. It has been shown, based on serial adoptive transfer experiments, spanning a few years of experimentation, that this cycle can be repeated more than 30 times without sizable diminution of the memory cell state (David B. Masopust, University of Minnesota, USA, “Repurposing Antiviral T Cells to Fight Tumors” at Keystone Symposium on Cancer Vaccines, Jan. 20-24, 2019, Fairmont Hotel Vancouver, Vancouver, British Columbia, Canada).

The “duality” of memory cells can be further described as follows: upon reactivation, memory cells mount a secondary rapid response and their progeny follow two fates: rapid generation of a new cohort of secondary (or tertiary if the second recall, or quaternary if a third recall) effectors (it is thought that the majority of reactivated memory cells follow this path) and self-renewal of memory cells which go on to preserve the memory pool (it is thought that this refers to a small proportion of the reactivated memory cells).

The amplitude of (re)activation may be characterized by an increased proliferation and/or production of pro-inflammatory and/or cytotoxic molecules/cytokines. For example, memory T-cells are capable of effectively producing the effector cytokines IFNγ and Tumor Necrosis Factor-α (TNF) and the proliferation-inducing cytokine IL-2 upon reencountering the specific antigen. This ability may be tested experimentally by restimulating the cells with Phorbol 12-Myristate 13-Acetate (PMA, a PKC activator) and Ionomycin (a Ca²+ ionophore, activating NF-κB and NFAT), as illustrated in the appended Examples and described previously (Tanchot (1998) 8(5):581-90).

It is also possible to restimulate memory T-cells by loading antigen-presenting cells with the cognate protein/peptide and co-culture those with the T-cells. However, such a restimulation assay depends on the previous (first) activation of the T-cells by the same cognate protein/peptide.

In contrast, restimulation with PMA and Ionomycin also works well for polyclonal T-cells, e.g. a pool of T-cells containing thousands of different TCRs recognizing at least as many different antigens. In other words, PMA and ionomycin allows for a non-TCR-specific global reactivation of the T-cells. Specifically, PMA and Ionomycin allow to (re)activate T-cells by providing the two signals that go downstream of the TCR signaling pathway, yet bypassing TCR engagement, i.e. PMA activates protein kinase C and Ionomycin is a calcium ionophore. PMA and Ionomycin together provide maximal activation of T-cells and/or B-cells.

Thus, the assays used herein, i.e. the restimulation with PMA and Ionomycin, allow to measure the effector functions of memory T-cells and/or T-cells with a memory phenotype, and the production of cytokines upon restimulation can thus be correlated with the memory differentiation state. In particular, if there are more T-cells with a memory phenotype present and/or the T-cells with a memory phenotype are more potent, they may give rise to more and/or more potent effector T-cells upon restimulation. Hence, the effector cytokine production, i.e. TNF, upon restimulation may be directly correlated with the quality of the memory-like T-cells before restimulation.

As described herein and demonstrated in the appended examples, the T-cells obtained by the method according to the invention, may be reactivated and respond with an increased magnitude/amplitude, i.e. when they have been generated and activated in the presence of an IDH2 inhibitor.

Thus, the cell population of the invention, i.e. the T-cells with a memory phenotype comprised therein, may have an unaltered or enhanced reactivation capacity. In particular, the reactivation capacity may be enhanced compared to the initial T-cells of the culture, e.g. naïve T-cells, and/or unaltered or enhanced compared to T-cells that have been generated the same way but without an IDH2 inhibitor and/or in the presence of an AKT inhibitor, as described herein.

A memory phenotype, as used herein, can also and/or additionally be defined by markers. Markers allow the distinction of a phenotype/cell state from another phenotype/cell state, for example a memory phenotype from an effector phenotype. A marker can be, for example, an RNA and/or protein whose presence or absence is associated with one or more important functional features. Such a marker may refer to the presence or absence of gene and/or protein expression and/or subcellular localization. This type of marker is typically described by “marker expression” or as “expression marker”. A marker can be also a functional property of a cell or cell population which can be determined by a standard assay. This type of marker is described as “functional marker”. Typical important functional features of a memory phenotype are mentioned herein, i.e. in the previous paragraph(s).

The presence or absence of a marker can be determined for a single cell and/or for a population of cells. Especially in case of a population of cells, suitable measurables are the average and median values and the frequency of cells with positive/negative and/or high/low values. For example, thresholds may be set and cells below a threshold may be considered “negative” for this marker (even if a numerically positive value is measured) and only cells above the threshold may be considered “positive”. More thresholds may be chosen to categorize cells, e.g., into low, mid and high marker expressing cells. This is further detailed out herein below. In some cases, a marker may also refer to another moment such as the variance.

Expression of a memory marker is associated with an important functional memory phenotype, whereas expression of a non-memory marker is associated with a cell state different from a memory phenotype. Absence or low expression of a memory marker is associated with a cell state different from a memory phenotype and absence or low expression of a non-memory marker may be associated with an important functional memory phenotype. Presence and/or high expression of a marker can be described, for example, by the symbols “+”, “⁺”, “high”, “positive”, “pos”. Intermediate marker expression can be described, for example, by the symbols “+/−”, “^{+/-}”, “mid”. Absence and/or low expression of a marker can be described, for example, by the symbols “−”, “⁻”, “low”, “negative”, “neg”. Typically, the terms “presence of”, “positive” and “high” marker expression are used interchangeably. Typically, the terms “absence of”, “negative” and “low” marker expression are used interchangeably. In cases, where exact distinction between positive and high, or negative and low expression is explicitly required, only the terms “high” or “low” refer to high or low expression, respectively.

Marker expression can be determined by methods well-known in the art, for example, but not limited to, flow cytometry, mass cytometry (also commonly known as CyTOF), western blot, quantitative RT-PCR, in-situ hybridization, microarray, RNA sequencing, nanostring, mass spectrometry, and fluorescent fusion-proteins expressed from an endogenous gene locus. Other synonymous terms for quantitative RT-PCR, as uses herein, are “Q-PCR”, “quantitative PCR”, “quantitative real time RT-PCR” and “RT Q-PCR”. The terms “flow cytometry” and “FACS” are used interchangeably herein in the context of marker analysis. A preferred method is flow cytometry/FACS.

To determine if the marker is expressed, the sample is preferably compared to a negative control. Preferably, the marker is not detected in the negative control. The threshold level for positive marker expression is preferably based on data from prior art and/or the present invention showing a link between said expression level and an important functional feature. Positive or negative marker expression cannot be solely based on the presence or absence of one or very few RNA or protein molecule(s). The threshold for positive marker expression, as used herein, does not depend on the detection method. Similarly, negative or low marker expression should be based on the expression level which is associated with a functional feature. A negative marker expression, as used herein, cannot be classified as positive only because a more sensitive detection method is used. For example, the expression of a marker at the cell surface can be determined by staining with a specific antibody and flow cytometry. When the staining intensity is similar to an unspecific staining, for example IgG isotype control staining, or autofluorescence (unstained) control, then it can be classified as negative or low. If a cell which has such a low level of marker expression has certain functional properties, then this may be a useful “negative” marker even when a more sensitive method would allow detecting some marker molecules in the cell. However, a sensitive detection method may allow determination of further useful markers.

Functional markers can be determined by a standard assay. Preferably, a standard assay is commercially available and comprises a detailed protocol. It may further, but not necessarily, comprise an internal reference. A standard assay is typically easier to perform than an assay for determining a complex inherent feature such as longevity in vivo. Preferably, a standard assay may be accomplished by routine in vitro experimentation.

Marker expression can occur and/or be measured at the cell surface or within a cell (intracellular). Marker expression can be measured in living cells and/or in dead (fixed) cells. Cell surface markers can typically be measured in living cells, for example by flow cytometry/FACS. Living FACS-sorted cells may be further used, for example, for in vitro culture and/or transferred into a subject.

Many memory markers are known in the art. Some memory markers may be similar between species and/or cell types. Other memory markers may be differentially expressed between species and/or cell types. Relevant species are, for example, mouse, human and non-human primates. Relevant cell types are, for example, CD8+ T-cells, CD4+ T-cells and B-cells. The memory phenotype, as used herein, is based on definitions used in the art. However, as the skilled person may know, those definitions may vary over time and between different laboratories/research groups/manufacturers. The more widely accepted a marker is and the more clearly and/or tightly it is linked to important functional properties of memory T-cells and/or B-cells, the more suitable this marker is.

The memory phenotype of T-cells, e.g. CD8+ T-cells, as used herein, refers primarily to stem cell memory T-cells (T_{SCM}) and/or central memory T-cells (T_{CM}). It does not refer to effector memory T-cells (T_{EM}), effector T-cells (T_{EFF}) and/or terminally differentiated T-cells (T_{EMRA}). The preferred T-cell memory phenotype, e.g. CD8+ T-cell memory phenotype, refers to central memory T-cells (T_{CM}).

Suitable positive expression markers for the memory phenotype (memory markers), in particular of CD8+ T-cells, but also, at least partly, of CD4+ T-cells and/or B-cells, are, for example, CCR7, CD62L, CD27, CD28, CD127 and/or TCF1.

Preferred memory (expression) markers are CCR7, CD62L and TCF1, in particular CD62L and TCF1, or CD62L and CCR7.

A very preferred memory (expression) marker is CD62L.

It has been further found in the context of the invention that an open chromatin configuration, e.g. in or in the vicinity of genes associated with the expression of the memory T-cell differentiation program, may be used as a marker of the memory phenotype as used herein, i.e. for T-cells. In particular, the open chromatin configuration may be characterized by an increased trimethylation on the lysine 4 residue of histone 3 (H3K4-3Me), an increased acetylation on lysine 27 residue of histone 3 (H3K27-Ac) and/or more accessible chromatin regions. The accessible chromatin regions may be determined by methods known in the art such as ATAC-seq, DNAse-Seq or MNase-Seq, e.g. as demonstrated in the appended Examples. H3K4-3 Me and/or H3K27-Ac levels and their associated genes may be also determined by methods known in the art, e.g. by western blotting, immunostaining, ChIP and/or ChIP-seq.

To further distinguish T-cells and/or B-cells with a memory phenotype from naïve T-cells and/or naïve B-cells, the auxiliary markers CD122, CD95 and/or production of IL-2 and/or IFN-gamma may be used. Those auxiliary markers, however, are not sufficient by themselves. A suitable negative expression marker for the memory phenotype (non-memory marker), in particular of CD8+ T-cells, but also, at least partly, of CD4+ T-cells and/or B-cells, is KLRG1.

A further suitable marker set for human central memory T-cells (comprised in “memory phenotype”) is: CCR7^{neg}/CD27⁺/CD28⁺/CD45RA^{neg}. Alternatively, the CD45RA expression may be low instead of negative. Furthermore, the human central memory T-cells may express CD45RO.

A suitable marker set for human central effector memory T cells (not comprised in “memory phenotype”) is: CCR7^{neg}/CD27^{+/-}/CD28^{+/-}/CD45RA^{neg}.

Suitable markers of T-cell differentiation, in particular memory markers and non-memory markers are also disclosed in Gattinoni et al., Nat Rev Cancer. 2012 Oct;12(10):671-84 and Kishton et al., Cell Metab. 2017 Jul 5;26(1):94-109.

Effector memory T-cells, effector T-cells and/or terminally differentiated T-cells, all which are not comprised in the term “memory phenotype” can be characterized by negative or low expression of CCR7, CD62L, CD27 and/or CD28, preferably in combination with positive or high expression of CD122, CD95 and/or KLRG1.

Effector T-cells are typically specialized for one or more specific functions, for example cytotoxicity, secretion of cytokines and/or activating or repressive modulation of other immune cells. Effector B-cells, including plasma cells, are typically specialized for the secretion of antibodies.

Naïve T-cells can be characterized by high or positive expression of CCR7, CD62L, CD27, CD45RA and/or CD28 and absence of CD122, CD95, IL-2R-beta and/or KLRG1. Naïve T-cells, e.g. from mouse, may also not express CD44. Furthermore, naïve T-cells are not cytotoxic and typically do not secret IL-2 and/or IFN-gamma. Naïve T-cells and/or B-cells have the capacity to differentiate into memory cells and/or effector cells. They have typically not been stimulated by antigen and a second stimulus. Naïve T-cells typically continuously recirculate between blood and the secondary lymphoid organs. It is in these organs that they may be presented with their cognate antigen by an antigen presenting cell following which they are activated. This event is also commonly referred to as “priming”. Following this event, they typically proliferate, acquire functional competencies, e.g. effector functions, and migrate to non-lymphoid tissues.

Thereby, naïve T-cells develop either into terminally differentiated short-lived effector cells (SLECs), or into memory precursor effector cells (MPECs) which can further differentiate into both, central memory T-cells (expressing CD62L) and effector memory T-cells (lacking expression of CD62L).

Central memory T-cells, descending from the memory precursor effector cells, typically reside, like naïve cells, in secondary lymphoid organs where they encounter the correct cytokine environment required for their self-renewal, however, a fraction also circulates in the blood. Upon re-exposure to antigen, these cells undergo activation, enhanced proliferation and rapid expression of effector functions. This event is commonly referred to as recall response or secondary response. While the majority of memory T-cells rapidly express effector functions, a variable but small fraction of them remains in the memory state of differentiation. This characteristic is called self-renewal. These are the dynamics of memory T-cells that are reminiscent of, and similar to, stem cells.

T-cells upregulate the central memory marker CD62L when cultured according to the invention. However, when the T-cells are transferred into a subject where they reencounter the antigen, they get reactivated. Upon (re-)activation in vivo, the T-cells quickly lose the CD62L marker, which however gets re-expressed when the T-cells differentiate into central memory T-cells again in vivo. Thus, the generation of memory precursor effector T-cells (MPECs) and/or central memory T-cells in vivo, for example in the spleen, upon transfer of the in vitro cultured T-cells into a subject, is a further characteristic of the memory phenotype. Further suitable functional memory markers may be based on metabolic changes which are known in the art to occur during the activation and/or differentiation of T-cells and/or B-cells. It is well known in the art that, for example, T-cells change from a mitochondrial metabolism and fatty acid oxidation towards an anabolic metabolism with aerobic glycolysis, see for example Kishton et al., Cell Metab. 2017 Jul 5;26(1):94-109. T-cells and/or B-cells with a memory phenotype can be distinguished from the respective effector cells by differences in oxygen consumption rates. In particular T-cells with a memory phenotype can be distinguished that way from effector memory T-cells (T_{EM}), effector T-cells (T_{EFF}) and/or terminally differentiated T-cells (T_{EMRA}).

T-cells and/or B-cells with a memory phenotype typically have an increased basal oxygen consumption, maximal respiratory capacity and/or spare respiratory capacity compared to the respective effector cells.

The basal oxygen consumption, maximal respiratory capacity and/or spare respiratory capacity can be measured, for example, with a XF-96 Extracellular Flux Analyzer (Seahorse Bioscience) in combination with suitable protocols and drugs. Suitable protocols are known in the art and can be obtained, for example, from Seahorse Bioscience. Addition of oligomycin allows to calculate the ATP-linked respiration by subtracting the oligomycin OCR from basal OCR. FCCP enables the electron transport chain (ETC) to reach its maximal rate, allowing to determine the cellular maximal respiratory capacity. Finally, rotenone and antimycin A inhibit the electron transport chain which indicates the non-mitochondrial respiration. The spare respiratory capacity (SRC) is defined as the difference between basal OCR and maximal OCR obtained after FCCP addition. The terms “basal oxygen consumption rate”, “basal oxygen consumption”, “basal respiration” and “basal OCR” are used interchangeably herein. The terms “maximal respiratory capacity”, “maximal respiration” and “MCR” are used interchangeably herein. The terms “spare respiratory capacity” and “SCR” are used interchangeably herein.

Activation of T-cells and/or B-cells typically occurs when an antigen is recognized by a TCR or BCR at the cell surface and usually only when a second stimulus is present. The second stimulus may be provided by APCs or other immune cells, in particular CD4+ helper T-cells and typically by additional protein-protein interactions at the cell surfaces. Typical second stimuli for T-cells provided by APCs are for example, 4-1BBL, CD80, CD86 and/or OX40L. Second stimuli for CD8+ T-cells may be provided by CD4+ T-cells, in particular helper T-cells. CD4+ T-cells and B-cells may provide each other with second stimuli.

Activation of T-cells and/or B-cells means that those cells differentiate and/or mature to perform specific tasks for eliminating an antigen and/or its source. Activation typically comprises proliferation of the cells and/or movement to a different tissue. During differentiation and/or maturation T-cells and/or B-cells change their phenotype. They also change their function. CD8+ T-cells may become cytotoxic, and CD4+ T-cells may differentiate into different subsets which may either support or repress the immune response. B-cells may secrete antibodies and undergo isotype switching. Differentiation into memory T-cells or memory B-cells may also occur. Activation usually involves secretion of cytokines to modulate the function of other immune cells.

Activation of T-cells and/or B-cells in vitro, as used herein, can be accomplished by methods known in the art, and may also refer to the “priming” of T-cells and/or B-cells. For example, T-cells may be activated by APCs, and/or by a mix of anti-CD3 and anti-CD28 antibodies. The anti-CD3 and anti-CD28 antibodies may be in solution, coupled to beads and/or attached to the surface of antigen presenting cells. In particular, the APCs may be used together with an antigenic peptide that is presented by the APCs.

Antigen-presenting cells that are particularly useful for the activation of T-cells may be dendritic cells, macrophages, B-cells, preferably dendritic cells, and/or artificial antigen presenting cells described in the art for this purpose (see, e.g., Neal (2017), J Immunol Res Ther. 2(1): 68-79).

Furthermore, the TCR and costimulatory receptor may be stimulated with anti-CD3/CD28 antibody beads, as illustrated in the appended Examples. Provision of a specific antigen, for example in case of OT1 T-cells, the ovalbumin N4 peptide (SIINFEKL) and/or provision of IL-2 may boost the activation/differentiation/maturation of T-cells and/or B-cells. In particular, the activation of T-cells, i.e. naïve T-cells, starts when the T-cells are contacted with an antigenic peptide (i.e. in combination with APCs), and/or a mix of anti-CD3 and anti-CD28 antibodies, and preferably IL-2. IL-2 may be present in solution or attached to the surface of antigen presenting cells. Evidently, the anti-CD3 and anti-CD28 antibodies must be suitable for stimulating the TCR and costimulatory receptor responsible for T-cell activation.

Furthermore, other molecules binding to said receptors may be also suitable in the context of the invention, if they stimulate T-cell activation.

In the in vitro culture method of the present invention, T-cell and/or B-cell activation and differentiation into memory T-cells and/or B-cells occur preferably side-by-side. Thus, the IDH2 inhibitor is preferably present in the culture medium together with the activating agent(s), i.e. an antigenic peptide (i.e. in combination with APCs), and/or a mix of anti-CD3 and anti-CD28 antibodies, and preferably IL-2.

The terms “differentiation” and “maturation”, as used herein, are not sharply separated. Differentiation, however, rather refers to cell states which maintain some plasticity, i.e. they may still give rise to different cell types, whereas the cell fate of cells which are maturing is more determined.

Suitable activation markers, as used herein, are CD25, CD44, CD71 and/or CD98. An additional activation marker is the extracellular acidification rate (ECAR) which can be measured by methods known in the art, for example with XF-96 Extracellular Flux Analyzer (Seahorse Bioscience) and/or in parallel with the oxygen consumption rate.

Suitable markers for the reactivation capacity of T-cells with a memory phenotype, i.e. upon reencounter of the antigen or a restimulation assay mimicking such a reencounter, as used herein, are IFNγ, TNF and/or IL2, preferably IFNγ. Since these markers are molecules that are normally secreted into the culture medium, a golgi inhibitor (e.g. Brefeldin A, BFA) can be applied during the restimulation assay. Application of a golgi inhibitor leads to the intracellular accumulation of these cytokines and thus allows for their measurement by flow cytometry or imaging. A normal, high or enhanced reactivation capacity, i.e. efficient expression of IFNγ, TNF and/or IL2, preferably IFNγ, may be used as a memory marker, i.e. a functional memory marker, as described herein.

Positive memory expression markers, negative memory expression markers, functional memory markers, and activation markers can be combined to describe a cell population obtained by a method of the invention. Furthermore, the cell population may be characterized by the chromatin configuration, i.e. whether it is in an open configuration, as described herein. The method of the invention allows maintaining a memory phenotype of T-cells and/or B-cells subjected to in vitro culture and/or allows generating T-cells and/or B-cells with a memory phenotype during in vitro culture. The term “generating” refers to differentiation from more naïve T-cells and/or B-cells. Expansion of the cells during in vitro culture is preferred, but not required.

Cells as used herein, when not specified explicitly or by context, refer to B-cells and/or T-cells. In a preferred embodiment of the invention, the cells are T-cells. In a very preferred embodiment, the T-cells comprise cytotoxic T-cells.

B-cells and T-cells are lymphocytes of the adaptive immune system. They are derived from hematopoietic stem cells. T-cells are important for cell-mediated immunity and B-cells are important for humoral immunity. Both are subjected to careful selection and tuning of their reactivity during ontogeny. The result is an immune system populated by naïve T-cells and B-cells which are largely depleted of autoreactive cells. There are two major subclasses of T-cells: CD4+ T-cells and CD8+ T-cells. CD8+ T-cells may develop into cytotoxic T-cells. Their major function is to destroy virus-infected cells and tumor cells. The terms “CD8+ T-cells” and “cytotoxic T-cells” are used herein interchangeably. Cytotoxic T-cells, as used herein, refer to CD8+ T-cells. As used herein, the term “cytotoxic T-cells” does not necessarily refer to an advanced differentiation stage but includes naïve CD8+ T-cells and/or memory CD8+ T-cells. CD4+ T-cells may, in some cases, also acquire cytolytic activity. There are various subsets of CD4+ T-cells. A major group of CD4+ T-cells are helper T-cells. Helper T-cells are often further grouped into different subsets of cells with specialized functions:

Th1: Produce an inflammatory response, key for defense against intracellular bacteria, viruses and cancer.

Th2: Aid the differentiation and antibody production by B cells.

Th17: Defense against gut pathogens and at mucosal barriers.

Th9: Defense against helminths.

Tfh: Help B cells produce antibody.

Another major group of CD4+ T-cells are regulatory T-cells. Regulatory T-cells are crucial for the maintenance of immunological tolerance. Their major role is to shut down T-cell-mediated immunity toward the end of an immune reaction and to suppress autoreactive T-cells that escaped the process of negative selection in the thymus.

Both, naïve CD8+ T-cells and CD4+ T-cells can differentiate into memory T-cells. There are different sets of memory T-cells which have in common that they are long-lived and can quickly expand to large numbers of effector T cells upon re-exposure to their cognate antigen. As used herein, memory T-cells or T-cells with a memory phenotype do not comprise effector memory T-cells, but rather refer to stem cell memory T-cells and/or central memory T-cells. A major function of B-cells is the generation of antibodies. Other functions include the presentation of antigens and production of cytokines. Memory B-cells are often dormant. Their function is to circulate through the body and initiate a stronger, more rapid antibody response (known as the anamnestic secondary antibody response) if they detect the antigen that had activated their parent B cell. Effector B-cells include plasmablasts and plasma cells. In certain embodiments, the cells may be a mix of CD8+ T-cells, CD4+ T-cells and/or B-cells. They may be mixed before, during or after in vitro culture and/or within a subject (before combined adoptive cell transfer and/or upon separate adoptive cell transfers). In certain embodiments, the cells are mammalian cells. In a preferred embodiment, the cells are human, non-human primate or mouse cells. In a very preferred embodiment the cells are human cells. In one embodiment, the cells are cells from pets such as cat cells or dog cells.

The terms “subject”, “patient” and “living organism” are used interchangeably herein and refer to a mammal, preferably a human, non-human primate or mouse, very preferably a human, which is the source of cells and/or the recipient of cells of the invention. Preferably, the subject is both source and recipient of the cells.

In certain embodiments, the memory phenotype comprises expression of one or more memory marker(s).

In certain embodiments, the memory phenotype comprises absence of expression of one or more non-memory marker(s).

Thus, the memory phenotype according to the invention, i.e. in the context of T-cells, preferably CD8+ T-cells, may comprise expression of at least one memory marker selected from the group consisting of: CD62L, TCF1, CD27, CD127, CCR7 and CD28. A further memory marker may be the nuclear localization of FOXO1. Furthermore, the memory phenotype may comprise absence of detectable expression of the non-memory marker KLRG1. Preferably, the memory phenotype comprises expression of the memory marker(s) CD62L and/or TCF1, preferably CD62L. Very preferably, the memory phenotype comprises surface expression of the memory marker CD62L.

In one embodiment, i.e. in the context of human T-cells, the memory phenotype comprises expression of CCR7, CD27, CD28 and no or low, preferably low, expression of CD45RA.

In certain embodiments, expression of memory marker(s) and/or expression of non-memory markers is compared to a DMSO control. In particular, this means that a memory marker is upregulated compared to the DMSO solvent control, and a non-memory marker is downregulated compared to the DMSO solvent control.

Furthermore, the T-cells according to the invention may express at least one activation marker, preferably wherein said at least one activation marker is selected from the group consisting of: CD25, CD44, CD71 and CD98.

Thus, in a particular embodiment, T-cells generated/maintained/obtained by a method of the present invention show surface expression of CD25, CD44, CD71, CD98 and increased surface expression of CD62L.

In certain embodiments, the memory phenotype comprises an increased basal oxygen consumption, maximal respiratory capacity and/or spare respiratory capacity compared to a DMSO control. Thus, the memory phenotype according to the invention i.e. in the context of T-cells, preferably CD8+ T-cells, may comprise an increased basal oxygen consumption, maximal respiratory capacity and/or spare respiratory capacity compared to the respective parameters in a control cell population, wherein the control T-cells have not been contacted with an IDH2 inhibitor. In particular, the basal oxygen consumption may be 1.1-fold to 1.5-fold, preferably about 1.3-fold, increased compared to a DMSO control. In particular, the maximal respiratory capacity may be 1.6-fold to 2-fold, preferably about 1.8-fold, increased compared to a DMSO control. In particular, the spare respiratory capacity may be 2-fold to 2.4-fold, preferably about 2.2-fold, increased compared to a DMSO control.

In certain embodiments, the extracellular acidification rate (ECAR) and/or the OCR/ECAR ratio is unchanged compared to a DMSO control. “Unchanged” means not significantly changed and/or having a very small effect size. Moreover, the ECAR of the T-cells with a memory phenotype according to the invention may be higher (e.g. 1.5-fold, 2-fold or 4-fold) than the ECAR of T-cells with a memory phenotyping obtained by in vitro culturing in the absence of an IDH2 inhibitor but in the presence of an AKT inhibitor, see Klebanoff (2017), loc.cit..

In certain embodiments, the memory phenotype comprises an open chromatin configuration as described herein. In particular, the open chromatin configuration may be characterized by an increased trimethylation on the lysine 4 residue of histone 3 (H3K4-3Me), an increased acetylation on lysine 27 residue of histone 3 (H3K27-Ac) and/or more accessible chromatin regions compared to the respective parameters in a control cell population, wherein the control T-cells have not been contacted with an IDH2 inhibitor.

In a preferred embodiment, the T-cells and/or B-cells are autologous cells. Autologous cells, as used herein, refer to cells which are derived from a subject which is to be treated with those cells, preferably after in vitro culture and/or manipulation.

In certain embodiments, the T-cells and/or B-cells comprise cells derived from tumor-infiltrating lymphocytes (TILs). Preferably, the TILs are T-cells. TILs may reside within a tumor of the patient which is to be treated with TILs with a memory phenotype obtained by the invention. Methods are known in the art to isolate TILs from the tumor of a patient. Isolated TILs are cultured by the method of the invention, in particular for adoptive cell transfer into said patient.

In one embodiment, the T-cells comprise T-cells that are derived from tumor-draining lymph node cells.

The term “derived”, as used herein, means that the cells have altered their cell state and/or environment/location, in particular that they are isolated from a subject and grown in vitro or ex vivo. Preferably, the derived T-cells maintain at least one characteristic from the T-cells from which they are derived, e.g. characteristic parts of the genome (such as the specific TCR gene), and/or the antigen specificity.

In certain embodiments, the T-cells and/or B-cells are derived from splenocytes and/or circulating blood cells. Further suitable sources of T-cells and/or B-cells are, for example, bone marrow, lymphoid organs, lymph, thymus and/or tissues infected by a parasite, bacteria and/or virus. For example, the T-cells and/or B-cells used for culturing according to the invention may be cells that have been obtained from the spleen, blood, e.g. cord blood or peripheral blood, a lymph node, i.e a tumor-draining lymph node, or from a tumor. Further suitable sources of T-cells and/or B-cells are induced pluripotent stem cells (iPSCs) as described in Temelli (2015), Cell Stem Cell 16(4):357-66 and/or in Nianias (2019), Curr Hematol Malig Rep 14(4):261-268.

The terms “in vitro” and “ex vivo” are used interchangeably herein and refer to the culture of cells in a culture dish, plate, flask and/or bottle outside of a living organism. Cell culture comprises a suitable liquid culture medium which allows the cells to survive and maintain and/or generate the desired phenotype and/or cell state. The cells may have been cultured and/or frozen previously. Preferably, the cells are freshly obtained from a subject before subjected to the in vitro culture of the invention.

A suitable cell culture medium for the culture of T-cells and/or B-cells is, for example, based on RPMI medium (basal medium). Preferably, said medium further comprises fetal-calf or human serum, Penicillin/Streptomycin, β-mercaptoethanol, HEPES, Non-essential amino acids, L-glutamine, Sodium Pyruvate, IL-2 and an antigenic peptide. Preferably, IL-7 is added during culture. Other media suitable for culturing T-cells and/or B-cells in cell manufacturing facilities may be based on commercially available basal media that limit the amount of fetal-calf or human serum while privileging inclusion of albumin, e.g. AIM V Serum Free Medium (Thermo Fisher Scientific).

For example, a suitable basal medium for culturing human T-cells may be RPMI medium supplemented with 10% human serum, penicillin (e.g. 50 IU/ml), streptomycin (e.g. 50 μg/ml), L-glutamine (e.g. 4 mM), non- essential amino acids (e.g. 1% (v/v)) and 2-mercaptoethanol (e.g. 50 μM).

Evidently, the culture medium according to the invention may comprise an IDH2 inhibitor as described herein.

Furthermore, the culture medium may further comprise further compounds according to the invention, e.g. IL-2, IL-7, an antigenic peptide, and/or anti-CD3 and anti-CD28 antibodies as described herein.

The meaning of adding a compound, for example an IDH2 inhibitor, IL-2, an antigenic peptide and/or IL-7, to the culture, culture medium and/or during in vitro culture, as used herein, is identical to contacting the cells with said compound during culture. As described herein, the compound may be added to the culture or culture medium at the beginning of the culture or after the culture has been initiated, preferably at the beginning of the culture. The terms “culture”, “cell culture”, “in vitro culture”, “culture medium” all refer to the in vitro or ex vivo environment where cells can be contacted with said compound, in particular an IDH2 inhibitor.

In a preferred embodiment, the T-cells and/or B-cells comprise naïve cells which acquire a memory phenotype.

In a preferred embodiment, the T-cells and/or B-cells are expanded during culture. Expansion means that more T-cells and/or B-cells exist at the end of the in vitro culture than at the start, e.g. as a result of cytokine-driven T-cell and/or B-cell proliferation.

In certain embodiments, the T-cells comprise a heterologous antigen receptor. The term “heterologous”, as used herein, refers to a gene or gene variant/allele which does not naturally occur in a certain cell. An antigen receptor as used herein, is a protein, typically expressed on the cell surface, which recognizes a specific antigen. A heterologous antigen receptor, as used herein, is thus a protein which binds to a specific antigen, which is expressed in a (host) cell, and which is encoded by a gene that has been introduced into said cell. Preferably the host cell is a T-cell or a B-cell. Very preferably, the host cell is a T-cell.

Preferably, the gene is stably integrated into the genome. Methods to stably integrate a gene into the genome of a host cell and to express this gene in that host cell are well known in the art. The recombinant DNA construct comprising the gene, in particular the gene encoding for a heterologous antigen receptor, further comprises a promoter which allows expression in the host cell. The promoter can be constitutively active, which means that it allows gene expression in nearly all cell types of the organism. A constitutive promoter is for example, but not limited to CAG, SFFV, PGK or CMV. The promoter can be also specific to the tissue of the host cell and/or related cells, for example cells into which the host cell can differentiate. A tissue specific promoter is, for example, a naturally occurring or modified promoter of a gene which is strongly expressed in said tissue(s). The recombinant DNA construct can be delivered by methods known in the art. The host cell can be, for example, electroporated, transfected, nucleofected and/or transduced with the recombinant DNA construct. Suitable transfection methods are, for example, based on lipofection, such as lipofectamine, or cationic polymers such as Polyethylenimine (PEI). The recombinant DNA construct may further comprise sequences for transposon mediated integration into the genome. When introduced together with a plasmid encoding the corresponding transposase into a host cell, the gene comprised in the recombinant DNA construct can be integrated into the genome of the host cell. Known transposon systems are, for example, piggyBAC or sleeping beauty. Preferably, the host cell is transduced with a viral vector comprising the recombinant DNA construct. Suitable transduction methods comprise, for example, retroviruses, lentiviruses, adenoviruses and/or adeno-associated viruses. The recombinant DNA construct may further comprise sequences required for virus production and/or integration of the gene into the genome of the host cell. Methods to generate viruses comprising the recombinant DNA construct, or an RNA variant thereof, are known in the art. Preferably, the virus, for example a lentivirus, mediates integration of the gene into the genome of the host cell.

In a preferred embodiment, the heterologous antigen receptor is a T-cell receptor (TCR). A specific heterologous TCR may be identical to a naturally occurring TCR and may or may not be expressed also naturally in the host cell, preferably the host T-cell. Typically, the heterologous TCR has been identified and/or selected by methods known in the art. Identification and/or selection methods comprise predicting and/or measuring the binding of the TCR to an MHC/HLA-antigen complex and/or isolating cells expressing a TCR. The antigen may be specific for the patient, for example for the tumor of a patient. A tumor specific antigen is also called TSA or neoantigen. The presence of an antigen may be also associated with a tumor, but not necessarily be tumor-specific. The interaction of a TCR with an MHC/HLA-peptide complex is typically patient specific, because the patient expresses a certain set of MHC/HLA alleles and often also specific antigens. A heterologous TCR is particularly useful for adoptive transfer of T-cells expressing this TCR into a patient. Particularly effective may be further vaccination with an antigen, for example an antigenic peptide or an antigen-presenting cell presenting the antigen by MHC/HLA molecules. Preferably, the T-cells are autologous T-cells which express a heterologous TCR that has been selected to efficiently bind to a patient-specific MHC/HLA-peptide complex comprising a patient-specific antigen, for example a neoantigen and/or a tumor-associated antigen.

In a preferred embodiment, the heterologous antigen receptor is a chimeric antigen-receptor (CAR). As used herein, a CAR refers to an artificial T-cell receptor. Chimeric antigen receptors combine many facets of normal T cell activation into a single protein. They link an extracellular antigen recognition domain to intracellular signaling domains, which activates the T cell when an antigen is bound. CARs are composed of three regions: the ectodomain, the transmembrane domain, and the endodomain.

The ectodomain is the region of the receptor that is exposed to the outside of the cell and so interacts with potential target molecules. It consists of 3 major components: an antigen recognition region that binds the target molecule, a signal peptide that directs the nascent protein into the endoplasmic reticulum, and a spacer that makes the receptor more available for binding. The antigen recognition region consists, for example, of a single-chain variable fragment (scFv). An scFv is a chimeric protein made up of the light (VL) and heavy (VH) chains of immunoglobins, connected with a short linker peptide. The transmembrane domain is a structural component, consisting of a hydrophobic alpha helix that spans the cell membrane, for example a CD28 transmembrane domain. The endodomain is the internal cytoplasmic end of the receptor that perpetuates signaling inside the T cell. The endodomain is, for example, based on CD3-zeta's cytoplasmic domain. The endodomain typically also includes one or more chimeric domains from co-stimulatory proteins such as CD28, 4-1BB (CD137), or OX40.

An IDH2 inhibitor, as used herein, is a compound or a mix of compounds which inhibits the enzyme Isocitrate dehydrogenase [NADP], mitochondrial (IDH2). Inhibition means that the function of the enzyme is blocked or impaired. In other words, inhibition of the enzyme means that its activity as catalyzer is impaired and/or less product is generated. Preferably, an IDH2 inhibitor inhibits the oxidative decarboxylation of isocitrate into alpha-ketoglutarate. Preferably, the inhibitor is specific to IDH2 which means that it does not inhibit or promote other enzymatic reactions. Preferably, the inhibitor does not promote an alternative enzymatic reaction of IDH2.

In some embodiments, the IDH2 inhibitor allosterically impairs the enzyme activity and/or disturbs the enzyme reaction in the active center of the enzyme, in particular by binding to the IDH2 protein.

In some embodiments, the IDH2 inhibitor may inhibit the production of the IDH2 protein, for example by inhibiting the transcription and/or translation of the idh2 gene and/or mRNA.

In a preferred embodiment, an IDH2 inhibitor inhibits the oxidative decarboxylation of isocitrate into alpha-ketoglutarate.

In a preferred embodiment, the concentration of the IDH2 inhibitor is high enough to inhibit the enzymatic reaction of wild-type IDH2. Preferably, the reaction of the wild-type IDH2 is the oxidative decarboxylation of isocitrate into alpha-ketoglutarate.

In some embodiments, the IDH2 inhibitor inhibits the formation of 2-hydroxyglutarate (2HG). In a preferred embodiment, the IDH2 inhibitor comprises one or more small molecule(s). A small molecule, as used herein, refers to a chemical compound, in particular an organic chemical compound, with low molecular weight, in particular less than 900 daltons.

Preferably, the small molecule IDH2 inhibitor inhibits the activity of IDH2.

In a very preferred embodiment, the small molecule(s) comprise(s) AG221 and/or AGI6780. In the most preferred embodiment, the small molecule comprises AG221.

AG221 or AG-221, as used herein, refers to CAS 1446502-11-9, Enasidenib and 2-methyl-1-[[4-[6-(trifluoromethyl)pyridin-2-yl]-6-[[2-(trifluoromethyl)pyridin-4-yl]amino]-1,3,5-triazin-2-yl]amino]propan-2-ol.

AGI6780 or AGI-6780, as used herein, refers to CAS 1432660-47-3 and 1-[5-(cyclopropylsulfamoyl)-2-thiophen-3-ylphenyl]-3-[3-(trifluoromethyl)phenyl]urea.

Preferably, AG221 and/or AGI6780 is administered orally. AG221 and/or AGI6780 may inhibit the wild-type IDH2 enzyme and/or specific mutant forms thereof, for example R140Q, R172S, and R172K.

In some embodiments, the IDH2 inhibitor blocks the enzyme activity of IDH2 by 20, 40, 60, 80 or 100%. Preferably, the enzyme activity is blocked by at least 60%. Very preferably, the enzyme activity is blocked by at least 80%.

Without being bound by theory, AG221 inhibits not only IDH2 mutants, but also the wild-type IDH2, when its concentration is high enough; Yen et al., Cancer Discov. 2017 May;7(5):478-493. However, the concentration should not be too high to avoid cytotoxic effects.

In a preferred embodiment, the concentration of concentration of AG221 and/or AGI6780 in the culture medium is about 1, 2, 3, 4 or 5 μM.

In a very preferred embodiment, the concentration of AG221 and/or AGI6780 in the culture medium is 5 μM.

Preferably, the concentration of AG221 and/or AGI6780 refers to the concentration of each of the two inhibitors.

In some embodiments, the IDH2 inhibitor comprises an oligonucleotide or a precursor thereof, which interferes with idh2 RNA. For example, the IDH2 inhibitor may comprise as oligonucleotide a small-interfering RNA (siRNA), a short-hairpin RNA (shRNA), an antisense RNA and/or the oligonucleotide functions by RNA-interference (RNAi), in particular to inhibit the production of IDH2 in a cell, for example by degrading the idh2 mRNA and/or inhibiting the translation of idh2 mRNA.

In some embodiments, the IDH2 inhibitor comprises an antibody and/or monobody. An (anti-IDH2) antibody and/or monobody, as used herein, inhibits the activity of IDH2 by binding to the IDH2 protein. Preferably, the anti-IDH2 antibody and/or monobody is a therapeutic antibody. Therapeutic antibodies are well known in the art, and may, for example, be humanized antibodies and/or have improved pharmacokinetic properties such as improved half-life if the blood plasma and or lead to enhanced clearance of IDH2. A monobody, as used herein, refers to a synthetic binding protein that is constructed using a fibronectin type III domain (FN3) as a molecular scaffold.

In some embodiments, the T-cells are contacted with the IDH2 inhibitor from the beginning of the culture and/or activation. In some embodiments, the T-cells are contacted with the IDH2 inhibitor during the entire culture period.

Preferably herein, the T-cells are contacted with the IDH2 inhibitor at least during activation, albeit it is not strictly required that the IDH2 inhibitor is present during the entire activation (priming) phase.

In some embodiments, the method of the invention further comprises a step of adding IL-2 to the culture. IL-2, as used herein, refers to interleukin-2.

In some embodiments, the method of the invention further comprises a step of adding one or more antigenic peptide(s) to the culture. Thus, the T-cells may be activated by contacting them with an antigenic peptide, in particular in the presence of antigen-presenting cells. An antigenic peptide, as used herein, is recognized by T-cells via a TCR and/or by B-cells via a BCR.

An antigenic peptide is, in particular, a short linear peptide, e.g. 8 to 25 amino acids in length, that can be presented by a MHC.

Typically, an antigenic peptide that is presented by a class I MHC molecule is 8 to 10 amino acids in length. Antigenic peptides that are presented by a class I MHC molecule may be particularly useful for generating and/or maintaining CD8+ T-cells with a memory phenotype according to the invention.

Typically, an antigenic peptide that is presented by a class II MHC molecule is 13 to 25 amino acids in length. Antigenic peptides that are presented by a class II MHC molecule may be particularly useful for generating and/or maintaining CD4+ T-cells with a memory phenotype according to the invention.

In one embodiment, the antigenic peptide(s)is/are specific for the activation of a specific T-cell, T-cell clone and/or group of T-cells.

Furthermore, the T-cells may be activated by contacting them with anti-CD3 and anti-CD28 antibodies.

B-cells may be also activated by antigens other than antigenic peptides, including proteins, haptens and any other molecule to which antibodies can be made to such as, inter alia, carbohydrates, lipids, or glycolipids.

IL-2 and/or an antigenic peptide may enhance the activation of T-cells and/or B-cells, in particular of T-cells, and/or their differentiation into cells with a memory phenotype. For example, adding IL-2 to the culture medium may promote cell expansion, production of IFN-gamma and/or cytotoxicity of CD8+ T-cells. The antigenic peptide may further promote selection of T-cells and/or B-cells which specifically recognize an antigen comprised in said peptide. Thus, the T-cells may be further contacted with IL-2, preferably together with the IDH2 inhibitor, preferably further together with the antigenic peptide and/or the anti-CD3 and anti-CD28 antibodies.

Thus, in a preferred embodiment, the T-cells are cultured in a medium comprising the IDH2 inhibitor, IL-2, and anti-CD3 and anti-CD28 antibodies and/or an antigenic peptide. B-cells may be cultured, and preferably expanded, in a medium comprising the IDH2 inhibitor and further a CD40 ligand, IL-2, IL-4, IL-10 and/or IL-21. Furthermore, for expanding B-cells, genetic modifications including “immortalization” may be employed, see e.g., Kwakkenbos (2016), Immunol Rev 270(1):65-77.

In some embodiments, the method of the invention further comprises a step of adding IL-7 to the culture medium. IL-7, as used herein, refers to interleukin-7.

Addition of IL-7 to the culture medium may promote the differentiation of B-cells and or T-cells into cells with a memory phenotype and/or the survival of the cells, in particular of cells with a memory phenotype (Raeber (2018) Immunol Rev 283(1):176-193). In particular, IL-7 may favor the survival of the T-cells in vivo upon adoptive transfer but IL-7 is not absolutely necessary for obtaining the T-cells with a memory phenotype upon IDH2 inhibition. Without being bound by theory, IL-7 induces glycerol channel AQP9 expression in CD8+ T cells which enhances triglyceride synthesis to promote memory CD8+ T cell survival; Cui et al., Cell. 2015 May 7;161(4):750-61.

In one embodiment, IL-7 is added to the culture medium after the IDH2 inhibitor and the antigenic peptide are washed out.

In a particular embodiment, the method further comprises the steps of
a. adding IL-2, an IDH2 inhibitor and an antigenic peptide to the culture medium for several days,
b. washing out the IDH2 inhibitor and the antigenic peptide and
c. adding IL-2 and IL-7 to the culture medium for several more days.

The time depends on the animal species and cells used. For example, when mouse CD8+ T-cells are used, step a of this embodiment takes about three days and step c about four days.

The skilled person is able to select suitable time periods for cells from other animal species, in particular for non-human primates or humans where biological processes such as cell differentiation take more time.

In other words, the T-cells may be cultured in a first medium comprising the IDH2 inhibitor, IL-2, and anti-CD3 and anti-CD28 antibodies and/or an antigenic peptide, and then in a second medium comprising IL-2 and IL-7. However, it is also possible that the cells are just cultured in said first medium, as described herein.

As already described above, the invention further relates to a cell population comprising T-cells with a memory phenotype obtained by the inventive method provided herein. Preferably, the T-cells are human cells. Accordingly, the method of the invention may be further characterized by producing the inventive cell population provided herein. The cell population of the invention can be produced by the inventive method provided herein, as demonstrated in the appended Examples. However, it remains possible that the inventive cell population can be also produced in the future by other methods yet to be developed, e.g. by modifying the inventive method provided herein. Evidently, the cell population of the invention is or can be isolated. In particular, a cell population obtained by an in vitro culture method is inherently separated from other cells by the culture vessel and may be kept separate from other cells.

As indicated above, it has been, inter alia, surprisingly found in the context of the present invention that a human cell population highly enriched for T-cells with a memory phenotype could be obtained by culturing a cell population comprising naive T-cells (e.g. human cord blood mononuclear cells) in the presence of an IDH2 inhibitor. As known in the art and described herein, T-cells with a memory phenotype are therapeutically more effective, when administered to a patient, compared to other T-cells, e.g. effector T-cells. However, it is not possible to obtain T-cells with a memory phenotype directly from patients, i.e. not at therapeutically effective numbers, and further manipulate them (e.g. transduction with a TCR or CAR) and put them back into a patient, without using a suitable in vitro culture method that promotes and/or maintains the memory phenotype of the cells, and preferably allows expansion of the cells. It is thus evident that an in vitro culture method for generating and/or maintaining T-cells with a memory phenotype is required for effectively using T-cells with a memory phenotype for therapy, i.e. immunotherapy. In other words, a cell population isolated from a subject/human comprising memory T-cells with a memory phenotype is not suitable for therapy without culturing the cell population in vitro. Thus, in practice, any cell population comprising T-cells with a memory phenotype, i.e. at a high frequency such as at least 30%, 40%, 50%, 60%, 70%, 80%, or 90%, is obtained by a method comprising in vitro culture of the cells. In other words, the inventive cell population provided herein is only obtainable by a method comprising in vitro culture. Moreover, the inventive cell population provided herein is suitable for use in therapy, and thus may be used for therapy, i.e. immunotherapy.

The cell population of the invention is, in particular, a cell population, wherein at least 90%, 95%, 98% or 99% of the cells in the population are T-cells, and/or at least 30%, 40%, 50%, 60%, 70%, 80%, or 90%, or at least 95%, 97%, 98% or 99%, preferably at least 70%, e.g. about 75%, of the cells in the population are T-cells with a memory phenotype. Preferably, said T-cells with a memory phenotype express CD62L. Preferably, the T-cells are human cells as described herein. The cell population comprising human T-cells with a memory phenotype according to the invention may be obtained after, e.g. 10 or 11 days, preferably 11 days, of culturing according to the inventive method provided herein.

The cell population of the invention may not be simply produced by enriching for T-cells with a memory phenotype obtained from another cell culture based on markers. Even if some markers, i.e. surface markers such as CD62L, could potentially allow to enrich for T-cells with a memory phenotype, such an approach may not allow to generate the cell population of the invention because the cells may be still functionally different (and/or different for the expression of other factors). Furthermore, merely enriching T-cells with a memory phenotype by sorting a culture comprising such cells based on memory marker expression (e.g. with FACS) may not provide a sufficiently large number of purified cells and may thus not be suitable for therapy, i.e. if T-cells with a memory phenotype only constitute a small proportion of the cells in the culture. Moreover, the sorting procedure usually leads to a cell loss for technical reasons which is counter the preferred aim to expand the T-cells with a memory phenotype, i.e. for therapeutic applications.

Thus, the cell population of the invention may be obtained by the inventive culture method provided herein without a cell sorting step such as FACS or MACS.

Thus, in one embodiment, the cell population of the invention is not purified based on marker expression, e.g. for CD62L positive T-cells, and/or not obtained by such a purification. In one embodiment, at least 90%, 95%, 98% or 99% of the cells in the inventive cell population provided herein are CD8+ T-cells.

In one embodiment, at least 90%, 95%, 98% or 99% of the cells in the inventive cell population provided herein are CD8+ T-cells or CD4+ T-cells.

Despite the fact that T-cells, CD4+ T-cells or CD8+ T-cells can be readily purified by methods known in the art, e.g. by using suitable antibodies and flow cytometry, the inventive cell population provided herein can be obtained, and is preferably obtained, without such further purification. In particular, only T-cells may proliferate when cultured according to the invention, while other cells that may be present such as antigen-presenting cells die and/or are diluted out towards the end of the culture.

However, in some embodiments, 90%, 95%, 98%, 99%, preferably 99.5% or 99.9% of the cells in the cell population are T-cells, CD8+ T-cells, or CD4+ T-cells, wherein such a cell population is obtained by further purifying the cell population obtained by the inventive in vitro culture method provided herein.

Moreover, the cell population of the invention may comprise a higher proportion of T-cells with a memory phenotype and/or show in average a more pronounced memory phenotype compared to a control cell population obtained in parallel by the same method except that the control T-cells have not been contacted with an IDH2 inhibitor.

Thus, the cell population may also refer to a population of T-cells and/or B-cells, which comprises, in particular, a higher proportion of T-cells and/or B-cells with a memory phenotype, as described herein.

Although various signaling pathways, i.e. PI3K signaling (Eid (2017) Cancer Res. 77(15):4135-4145), rapamycin/mTOR/RICTOR signaling (Araki (2009) Nature 460(7251):108-12; Scholz (2016) EBioMedicine 4:50-61; Li (2012) J Immunol 188(7):3080-7; Zhang (2016) Cell Rep 14(5):1206-1217; Pollizzi (2015) J Clin Invest 125(5):2090-10), and WNT signaling (Muranski (2011) Immunity 35(6):972-85), or AMPK activation (Pearce (2009) Nature 460(7251):103-7) have been implicated in promoting a memory phenotype in human T-cells, the only practical in vitro culture protocol which has been suggested to be useful for the generation and/or maintenance of human T-cells with a memory phenotype, i.e. for the induction of CD62L expression, employs an AKT inhibitor (Klebanoff et al., JCI Insight. 2017 Dec 7;2(23):e95103). Furthermore, AKT inhibition, PI3K inhibition or mTOR inhibition may negatively interfere with the activation of T-cells and not provide an optimal yield, as described above. Moreover, the inventors of the present invention have found that the memory-like T-cells generated by in vitro culture with an AKT inhibitor had a reduced capacity of expressing the effector cytokine IFNγ upon restimulation compared to control T-cells or T-cells contacted with an IDH2 inhibitor according to the present invention. In other words, the human memory-like T-cells according to Klebanoff (2017) loc. cit., seem to have a deficit in an important functional feature associated with memory T-cells, namely the ability to react with an increased amplitude to a reencounter of an antigen, whereas this ability seems to be unaltered or enhanced in the inventive T-cells with a memory phenotype provided herein. Furthermore, the inventive T-cells with a memory phenotype provided herein may have a normal ECAR (as demonstrated for the mouse cells in the appended Examples), whereas the ECAR is reduced in the T-cells with a memory phenotype obtained by AKT inhibition (instead of IDH2 inhibition; see Klebanoff (2017) loc. cit.).

This further demonstrates that the cell population comprising human T-cells with a memory phenotype obtained by the inventive method provided herein (the human cell product or T-cell product of the invention) is different from the cell populations disclosed in Klebanoff (2017) loc. cit.. Furthermore, the inventive cell population provided herein, may be characterized, for example, by an increased percentage of cells expressing CD62L on the membrane, increased trimethylation on the lysine 4 residue of histone 3 (H3K4-3Me), increased acetylation on lysine 27 residue of histone 3 (H3K27-Ac), and/or alterations in the chromatin conformation resulting in more accessible regions (e.g. more than 1000, such as 1633).

Thus, the T-cells of the invention, i.e. in the context of the inventive method and/or cell population provided herein, may have an unaltered or enhanced reactivation capacity, in particular an unaltered or enhanced capacity of producing IFNγ upon restimulation. Preferably, said T-cells are CD8+ T-cells. In particular, the capacity of the herein provided T-cells with a memory phenotype to produce IFNγ upon restimulation may be unaltered or enhanced compared to (seemingly) corresponding T-cells with a memory phenotype that have been obtained by in vitro culture in the absence of an IDH2 inhibitor. Corresponding T-cells may appear similar to the T-cells of the invention, e.g. for the expression of some markers such as CD62L, and/or may be obtained by a culture method which is similar except for the contacting with an IDH2 inhibitor. However, said (seemingly) corresponding T-cells may in fact be different in one or more characteristic, e.g. the reactivation capacity as described herein.

In particular, said restimulation may comprise contacting the T-cells with a memory phenotype with Phorbol 12-Myristate 13-Acetate and Ionomycin.

Moreover, the T-cells with a memory phenotype according to the invention may have an enhanced capacity to produce IFNγ upon restimulation compared to T-cells with a memory phenotype that are obtained by in vitro culture in the absence of an IDH2 inhibitor but in the presence of an AKT inhibitor.

Furthermore, the T-cells with a memory phenotype according to the invention may have a higher expression of activation markers such as CD44, CD71 and/or CD98 compared to T-cells with a memory phenotype that are obtained by in vitro culture in the absence of an IDH2 inhibitor but in the presence of an AKT inhibitor.

Thus, in one embodiment. the T-cells are not contacted with an AKT inhibitor and/or have not been contacted with an AKT inhibitor.

In the context of the invention, at least 35% of the cells in the inventive cell population provided herein may be human CD8+ T-cells with a memory phenotype that express CD62L, in particular, wherein said CD8+ T-cells have not been contacted with an AKT inhibitor.

In a particular embodiment, at least 60%, 70%, 80%, or 90%, preferably at least 70%, e.g. about 75%, of the human CD8+ T-cells in the inventive cell population provided herein are T-cells with a memory phenotype that express CD62L, in particular, wherein said CD8+ T-cells have not been contacted with an AKT inhibitor.

In a further particular embodiment,
(a) at least 90%, 95%, 98% or 99% of the cells in the inventive cell population provided herein are human T-cells, and/or
   at least 35% of the cells in the inventive cell population provided herein are human CD8+ T-cells that express CD62L,
(b) at least 60%, 70%, 80%, or 90%, preferably at least 70%, e.g. about 75% of the human CD8+ T-cells in the inventive cell population provided herein express CD62L and/or, the percentage of human CD8+ T-cells that express CD62L and/or the average CD62L expression of the human CD8+ T-cells is greater than in a control cell population comprising human CD8+ T-cells, wherein said control cell population has been obtained by in vitro culture in the absence of an IDH2 inhibitor, and
(c) the human CD8+ T-cells have an enhanced capacity to produce IFNγ upon restimulation with Phorbol 12-Myristate 13-Acetate and Ionomycin compared to
   (i) the CD8+ T-cells in said control cell population, and
   (ii) CD62L expressing human CD8+ T-cells that have been obtained by in vitro culture in the absence of an IDH2 inhibitor and in the presence of an AKT inhibitor.

Furthermore, the T-cells of the invention, i.e. comprised in the cell population of the invention may maintain a memory phenotype in vivo when administered to a subject, as demonstrated in the appended Examples. In particular, the T-cells may efficiently give rise to memory precursor effector T-cells (MPECs) and/or central memory T-cells in vivo, for example in the spleen, when administered to a subject, in particular upon reencounter of the antigen. In particular, said capability or efficiency may be increased compared to T-cells generated by an in vitro culture method which does not employ an IDH2 inhibitor but is preferably otherwise

identical, e.g. a DMSO control, as described herein. In one embodiment, the T-cells cultured and/or contacted with the IDH2 inhibitor are human cord blood mononuclear cells (mononuclear cells from umbilical cord blood; CBMCs), or peripheral blood mononuclear cells (PBMCs).

Furthermore, the present invention relates to an in vitro cell culture comprising the inventive cell population provided herein. In particular, said in vitro cell culture may further comprise an IDH2 inhibitor as described herein. Moreover, said in vitro cell culture may comprise a culture medium described herein in the context of the inventive method.

In one embodiment, the inventive method provided herein comprises a step of transferring cultured T-cells and/or B-cells into a subject.

Thus, the cell population of the invention may be used in therapy, i.e. immunotherapy, in particular wherein the cell population or the T-cells comprised in said cell population is/are administered to a patient.

The term “immunotherapy”, as used herein, refers to the treatment of a disease by modulating (activating or suppressing) the immune system. The term “immunotherapy” comprises modulation of the immune system in a subject (in vivo treatment) and/or transferring immune cells which have been modulated during in vitro culture into a subject (cell-based immunotherapy). Preferably, the immunotherapy is a cell-based immunotherapy.

Accordingly, the invention relates to an IDH2 inhibitor for use in immunotherapy. Preferably, said immunotherapy comprises administering T-cells to a patient, wherein the T-cells have been contacted with the IDH2 inhibitor during in vitro culture according to the method of the invention, in particular wherein said T-cells have thereby acquired a memory phenotype in vitro.

Accordingly, the invention relates to a population of cells obtained by the method of the invention for use in immunotherapy.

Accordingly, the invention relates to an immunotherapy comprising administering an IDH2 inhibitor to a patient.

Accordingly, the invention relates to an immunotherapy comprising administering T-cells and/or B-cells contacted with an IDH2 inhibitor to a patient. In particular, the cells are contacted with an IDH2 inhibitor during in vitro culture.

Accordingly, the invention also relates to a method for generating and/or maintaining T-cells and/or B-cells with a memory phenotype in a subject comprising administering an IDH2 inhibitor to said subject.

In certain embodiments, the immunotherapy comprises T-cells and/or B-cells which acquire or have acquired a memory phenotype within a subject, in vitro and/or ex vivo.

In a preferred embodiment, the immunotherapy comprises T-cells and/or B-cells which have acquired a memory phenotype in vitro.

In a preferred embodiment, the immunotherapy comprises adoptive cell transfer. Adoptive cell transfer (ACT), as used herein, refers to the transfer of cells into a patient. The cells may have originated from the patient or from another individual. Preferably, the cells have originated from the patient (autologous cells). Preferably, the cells are extracted from the patient, cultured in vitro and returned to the same patient. Alternatively, the cells are isolated and expanded from a donor separate from the patient receiving the cells. The terms “adoptive cell transfer”, “ACT”, “adoptive transfer” and “cells adoptively transferred” are used interchangeably herein. During in vitro culture, the cells may be genetically modified. Preferably, the cells are genetically modified by integrating a TCR or CAR into the genome. The cells may be also modified to inhibit an intrinsic checkpoint. For example, Cytokine-inducible SH2-containing protein (CISH) may be knocked-out or knocked-down in T-cells. Furthermore, the T-cells may be modified to knock out the endogenous TCR in the case of T-cells isolated and expanded from a donor separate from the recipient patient (i.e. in allogeneic T-cells).

In a preferred embodiment, the immunotherapy comprises adoptive transfer of T-cells and/or B-cells which have acquired a memory phenotype in vitro into a patient.

In a preferred embodiment, the immunotherapy is a T-cell therapy. A T-cell therapy, as used herein, refers to the modulation of T-cells in a subject and/or the adoptive transfer of in vitro cultured T-cells into a patient. In a very preferred embodiment, the T-cells therapy comprises adoptive transfer of in vitro cultured T-cells into a patient. Preferably, the cultured and adoptively transferred T-cells are CD8+ T-cells, preferably autologous CD8+ T-cells.

In particular, the T-cells may comprise CD8+ T-cells, the T-cells may be autologous cells, and/or the T-cells may be derived from tumor-infiltrating T-cells.

In one embodiment, the T-cells comprise allogeneic T-cells. For example, the allogeneic T-cells may be, inter alia, from umbilical cord blood or peripheral blood. Furthermore, the endogenous TCR of the allogeneic T-cells may be knocked out.

In a preferred embodiment, the immunotherapy is a therapy to treat cancer. An immunotherapy to treat cancer, as used herein, may activate the immune system to contain and/or eliminate cancer cells. The terms “tumor”, “cancer”, “tumor cells” and “cancer cells” are used interchangeably herein and comprise benign and malign tumors as well as single cancer cells, solid tumors, liquid tumors, circulating tumor cells, clusters of cancer cells and metastases. Preferably, the term “cancer” refers to a malign tumor. The invention is not limited to the treatment of a specific type of cancer. Preferably, an IDH2 inhibitor and/or a population of cells obtained by the method of the invention, may be used for the treatment of melanoma, other solid tumors, and/or hematological malignancies, for example leukemia. The invention may be particularly useful for the treatment of late-stage and/or aggressive cancers, for example metastatic cancer and/or cancer that is resistant to other cancer therapies. In a preferred embodiment, the cancer is resistant to chemotherapy, targeted therapy and/or antibody-mediated immunotherapy.

Chemotherapy, as used herein, refers to a type of cancer treatment that uses one or more anti-cancer drugs (chemotherapeutic agents) as part of a standardized chemotherapy regimen. Typically, a chemotherapeutic agent inhibits cell division. Typical chemotherapeutic agents are, for example, but not limited to, Chlorambucil, Valrubicin, Abraxane, Vorinostat, Irinotecan, Etoposide, Bortezomib, Vemurafenib, Fluorouracil, Actinomycin, Oxaliplatin, Tretinoin and Vinblastine.

Targeted therapy, as used herein, refers to blocking the growth of cancer cells by interfering with specific targeted molecules needed for carcinogenesis and tumor growth. Typically, compounds used for targeted therapy are small molecules and/or monoclonal antibodies. Typical small molecules for targeted therapy are, for example, but not limited to Imatinib, Erlotinib, Vemurafenib, Everolimus, Obatoclax, Crizotinib, Sunitinib. Typical monoclonal antibodies for targeted therapy are, for example, but not limited to Rituximab, Trastuzumab, Bevacizumab and Cetuximab.

In some cases, a small molecule can be classified both for use in chemotherapy and targeted therapy. In contrast, a small molecule which is known for use in chemotherapy and/or targeted therapy cannot be predicted to have an effect in immunotherapy. Chemotherapy and/or targeted therapy usually act directly on cancer cells, whereas immunotherapy acts primarily on immune cells. Monoclonal antibodies, however, may be classified both for use in targeted therapy and immunotherapy, because they may, in contrast to small molecules, both interfere with a cancer-associated molecule and stimulate the immune system.

The term “antibody mediated immunotherapy”, as used herein, refers to monoclonal antibodies which modulate and/or stimulate the immune system, for example through Antibody-dependent cell-mediated cytotoxicity (ADCC), the complement system and/or blocking immunosuppressive mechanisms (checkpoints) such as the PD-1/PD-L1 interaction. Typical monoclonal antibodies for targeted therapy are, for example, but not limited to Alemtuzumab, Durvalumab, Nivolumab, Pembrolizumab, Trastuzumab, Pertuzumab, Monalizumab and Rituximab.

In a preferred embodiment, the cancer comprises metastases.

In some embodiments, the cell population of the invention and/or T-cells with a memory phenotype according to the invention is/are administered to a patient in combination with an additional anti-cancer drug, preferably a checkpoint inhibitor, as described herein, i.e. in the context of the composition comprising an IDH2 inhibitor. The additional anti-cancer drug may be administered before, concomitantly and/or after administration of the cell population/T-cells of the invention.

In one embodiment, the immunotherapy comprises administering an IDH2 inhibitor to a subject. Preferably, the IDH2 inhibitor is administered to a subject for the treatment of cancer. The invention also relates to a composition comprising an IDH2 inhibitor for use in immunotherapy.

In one embodiment, a composition for use in the treatment of cancer comprises at least one additional anti-cancer drug. Said anti-cancer drug(s) may be selected from a chemotherapeutic agent, an agent for targeted therapy and/or a monoclonal antibody for antibody mediated immunotherapy, as described herein.

In a preferred embodiment, the composition for use in the treatment of cancer further comprises a checkpoint inhibitor, for example a molecule which targets CTLA4, PD-1, and/or PD-L1, such as, but not limited to, Ipilimumab, Pembrolizumab, Nivolumab, Atezolizumab and Avelumab.

In some embodiments, the immunotherapy is a therapy to treat a chronic viral infection. Preferably, the chronic viral infection is HIV. Without being bound by theory, a long-lasting pool of HW specific T-cells and/or B-cells with a memory phenotype may increase the chance of eradicating HIV infected cells.

In some embodiments, the immunotherapy is a therapy to treat an autoimmune disease. Autoimmune diseases are, for example, but not limited to celiac disease, diabetes mellitus type 1, Graves' disease, inflammatory bowel disease, multiple sclerosis, psoriasis, rheumatoid arthritis, and systemic lupus erythematosus. Preferably, the cells used for treating an autoimmune disease are regulatory T-cells with a memory phenotype. Such long-lasting regulatory T-cells may be used to protect a patient against aberrant immune responses; Rosenblum et al.; Nat Rev Immunol. 2016 Feb;16(2):90-101. Additionally, cytotoxic T-cells and/or helper T-cells with a memory phenotype and/or antibodies derived from B-cells with a memory phenotype may recognize specific T-cells and/or B-cells which contribute to an autoimmune reaction and cause or contribute to the elimination of the latter.

As used herein, “treatment” (and grammatical variations thereof such as “treat” or “treating”) refers to clinical intervention in an attempt to alter the natural course of the individual being treated. Desirable effects of treatment include, but are not limited to, prophylaxis, preventing occurrence or recurrence of disease or symptoms associated with disease, alleviation of symptoms, diminishment of any direct or indirect pathological consequences of the disease, decreasing the rate of disease progression, amelioration or palliation of the disease state, improved prognosis and cure.

Furthermore, the invention relates to:
1. A method for generating and/or maintaining T-cells and/or B-cells with a memory phenotype comprising the steps of
   a. culturing T-cells and/or B-cells in vitro and
   b. adding an IDH2 inhibitor to the culture of a).
2. The method of embodiment 1, further comprising a step of obtaining T-cells and/or B-cells with a memory phenotype from the culture.
3. The method of embodiments 1 or 2, wherein the T-cells and/or B-cells are activated before and/or during culture.

4. The method of any one of embodiments 1 to 3, wherein the cells are T-cells.

5. The method of any one of embodiments 1 to 4, wherein the T-cells comprise cytotoxic T-cells.

6. The method of any one of embodiments 1 to 5, wherein the T-cells and/or B-cells are human, non-human primate or mouse cells.

7. The method of any one of embodiments 1 to 6, wherein the memory phenotype comprises expression of one or more memory marker(s).

8. The method of any one of embodiments 1 to 7, wherein the memory phenotype comprises expression of the memory marker(s) CD62L and/or TCF1.

9. The method of any one of embodiments 1 to 8, wherein the memory phenotype comprises surface expression of the memory marker CD62L.

10. The method of any one of embodiments 1 to 9, wherein the memory phenotype comprises surface expression of the memory marker(s) CD27 and/or CD127.

11. The method of any one of embodiments 1 to 10, wherein the memory phenotype comprises expression of the memory marker(s) CCR7, CD27 and/or CD28.

12. The method of any one of embodiments 1 to 11, wherein the memory phenotype comprises absence of detectable surface expression or low surface expression of the non-memory marker KLRG1.

13. The method of any one of embodiments 1 to 12, wherein the memory marker(s) is/are upregulated compared to a DMSO control.

14. The method of any one of embodiments 1 to 13, wherein the non-memory marker(s) is/are downregulated compared to a DMSO control.

15. The method of any one of embodiments 1 to 14, wherein the T-cells and/or B-cells express one or more activation marker(s).

16. The method of any one of embodiments 1 to 15, wherein the T-cells show surface expression of the activation marker(s) CD25, CD44, CD71 and/or CD98.

17. The method of any one of embodiments 1 to 16, wherein the T-cells show surface expression of CD25, CD44, CD71, CD98 and increased surface expression of CD62L.

18. The method of any one of embodiments 1 to 17, wherein the memory phenotype comprises an increased basal oxygen consumption, maximal respiratory capacity and/or spare respiratory capacity compared to a DMSO control.

19. The method of any one of embodiment 18, wherein the basal oxygen consumption is about 1.3-fold increased compared to a DMSO control.

20. The method of embodiments 18 or 19, wherein the maximal respiratory capacity is about 1.8-fold increased compared to a DMSO control.

21. The method of any one of embodiments 18 to 20, wherein the spare respiratory capacity is about 2.2-fold increased compared to a DMSO control.

22. The method of any one of embodiments 1 to 21, wherein the T-cells and/or B-cells are autologous cells.

23. The method of any one of embodiments 1 to 22, wherein the T-cells and/or B-cells comprise T-cells derived from tumor-infiltrating lymphocytes.

24. The method of any one of embodiments 1 to 23, wherein the T-cells and/or B-cells comprise T-cells and/or B-cells derived from splenocytes.

25. The method of any one of embodiments 1 to 24, wherein the T-cells and/or B-cells comprise naïve cells which acquire a memory phenotype.

26. The method of any one of embodiments 1 to 25, wherein the T-cells and/or B-cells are expanded during culture.

27. The method of any one of embodiments 1 to 26, wherein the T-cells comprise a heterologous antigen receptor.

28. The method of embodiment 27, wherein the heterologous antigen receptor is a TCR.

29. The method of embodiment 27, wherein the heterologous antigen receptor is a CAR.

30. The method of any one of embodiments 1 to 29, wherein the IDH2 inhibitor comprises one or more small molecule(s).

31. The method of embodiment 30, wherein the small molecule(s) comprise(s) AG221 and/or AGI6780, and wherein AG221 is 2-methyl-1-[[4[6-(trifluoromethyl)pyridin-2-yl]-6-[[2-(trifluoromethyl)pyridin-4-yl]amino]-1,3,5-triazin-2-yl]amino]propan-2-ol and AGI6780 is 1-[5-(cyclopropylsulfamoyl)-2-thiophen-3-ylphenyl]-3-[-3-(trifluoromethyl)phenyl]urea.

32. The method of embodiment 31, wherein the concentration of AG221 and/or AGI6780 is about 1, 2, 3, 4 or 5 μM.

33. The method of embodiment 31 or 32, wherein the concentration of AG221 and/or AGI6780 is about 5 μM.

34. The method of any one of embodiments 1 to 33, wherein, the IDH2 inhibitor comprises a nucleotide or a precursor thereof, which interferes with idh2 RNA.

35. The method of any one of embodiments 1 to 34, wherein the IDH2 inhibitor comprises an antibody and/or monobody.

36. The method of any one of embodiments 1 to 35, further comprising a step of adding IL-2 to the culture.

37. The method of any one of embodiments 1 to 36, further comprising a step of adding one or more antigenic peptide(s) to the culture.

38. The method of embodiment 37, wherein the antigenic peptide(s) is/are specific for the activation of a specific T-cell, T-cell clone and/or group of T-cells.

39. The method of any one of embodiments 1 to 38, further comprising a step of adding IL-7 to the culture medium.

40. The method of any one of embodiments 1 to 39, further comprising the steps of
a. adding IL-2, an IDH2 inhibitor and an antigenic peptide to the culture medium for several days,
b. washing out the IDH2 inhibitor and the antigenic peptide and
c. adding IL-2 and IL-7 to the culture medium for several more days.

41. The method of any one of embodiments 1 to 40, further comprising a step of transferring the cultured T-cells and/or B-cells into a subject.

42. An IDH2 inhibitor for use in immunotherapy.

43. The IDH2 inhibitor for use according to embodiment 42, wherein the immunotherapy comprises T-cells and/or B-cells which acquire or have acquired a memory phenotype in vivo and/or ex vivo.

44. The IDH2 inhibitor for use according to embodiments 42 or 43, wherein the immunotherapy is a T-cell therapy.

45. The IDH2 inhibitor for use according to any one of embodiments 42 to 44, wherein the IDH2 inhibitor comprises one or more small molecule(s).

46. The IDH2 inhibitor for use according to embodiment 45, wherein the small molecule(s) comprise(s) AG221 and/or AGI6780 and wherein AG221 is 2-methyl-1-[[4-[6-(trifluoromethyl)pyridin-2-yl]-6-[[2-(trifluoromethyl)pyridin-4-yl]amino]-1,3,5-triazin-2-yl]amino]propan-2-ol and AGI6780 is 1-[5-(cyclopropylsulfamoyl)-2-thiophen-3-ylphenyl]-3-[3-(trifluoromethyl)phenyl]urea.

47. The IDH2 inhibitor for use according to embodiment 46, wherein the concentration of AG221 and/or AGI6780 is about 1, 2, 3, 4 or 5 μM.

48. The IDH2 inhibitor for use according to embodiments 46 or 47, wherein the concentration of AG221 and/or AGI6780 is about 5 μM. 49. The IDH2 inhibitor for use according to any one of embodiments 42 to 48, wherein the IDH2 inhibitor comprises a nucleotide or a precursor thereof, which interferes with idh2 RNA.

50. The IDH2 inhibitor for use according to any one of embodiments 42 to 49, wherein the IDH2 inhibitor comprises an antibody and/or monobody.

51. The IDH2 inhibitor for use according to one of embodiments 42 to 50, wherein the immunotherapy is a therapy to treat cancer.

52. The IDH2 inhibitor for use according to embodiment 51, wherein the cancer is resistant to chemotherapy, targeted therapy and/or antibody-mediated immunotherapy.

53. The IDH2 inhibitor for use according to embodiments 51 or 52, wherein the cancer comprises metastases.

54. The IDH2 inhibitor for use according to any one of embodiments 42 to 50, wherein the immunotherapy is a therapy to treat a chronic viral infection.

55. The IDH2 inhibitor for use according to any one of embodiments 42 to 50, wherein the immunotherapy is a therapy to treat an autoimmune disease.

56. The IDH2 inhibitor for use according to any one of embodiments 43 to 55, wherein the T-cells and/or B-cells are human, non-human primate or mouse cells.

57. The IDH2 inhibitor for use according to any one of embodiments 43 to 56, wherein the T-cells and/or B-cells are autologous cells.

58. The IDH2 inhibitor for use according to any one of embodiments 43 to 57, wherein the T-cells comprise cytotoxic T-cells.

59. The IDH2 inhibitor for use according to any one of embodiments 43 to 58, wherein the T-cells and/or B-cells comprise tumor-infiltrating lymphocytes.

60. The IDH2 inhibitor for use according to any one of embodiments 43 to 59, wherein the T-cells comprise a heterologous antigen receptor.

61. The IDH2 inhibitor for use according to embodiment 60, wherein the heterologous antigen receptor is a TCR.

62. The IDH2 inhibitor for use according to embodiment 60, wherein the heterologous antigen receptor is a CAR.

63. The IDH2 inhibitor for use according to any one of embodiments 43 to 62, wherein most or nearly all T-cells and/or B-cells have a memory phenotype.

64. The IDH2 inhibitor for use according to any one of embodiments 43 to 63, wherein the memory phenotype comprises expression of one or more memory marker(s).

65. The IDH2 inhibitor for use according to any one of embodiments 43 to 64, wherein the memory phenotype comprises expression of the memory marker(s) CD62L and/or TCF1.

66. The IDH2 inhibitor for use according to any one of embodiments 43 to 65, wherein the memory phenotype comprises surface expression of the memory marker CD62L.

67. The IDH2 inhibitor for use according to any one of embodiments 43 to 66, wherein the memory phenotype comprises surface expression of the memory marker(s) CD27 and/or CD127.

68. The IDH2 inhibitor for use according to any one of embodiments 43 to 67, wherein the memory phenotype comprises expression of the memory marker(s) CCR7, CD27 and/or CD28.

69. The IDH2 inhibitor for use according to any one of embodiments 43 to 68, wherein the memory phenotype comprises absence of detectable surface expression or low surface expression of the non-memory marker KLRG1.

70. The IDH2 inhibitor for use according to any one of embodiments 43 to 69, wherein the memory marker(s) is/are upregulated compared to a control.

71. The IDH2 inhibitor for use according to any one of embodiments 43 to 70, wherein the non-memory marker(s) is/are downregulated compared to a control.

72. The IDH2 inhibitor for use according to any one of embodiments 43 to 71, wherein the T-cells and/or B-cells express one or more activation marker(s).

73. The IDH2 inhibitor for use according to any one of embodiments 43 to 72, wherein the T-cells show surface expression of the activation marker(s) CD25, CD44, CD71 and/or CD98.

74. The IDH2 inhibitor for use according to any one of embodiments 43 to 73, wherein the T-cells show surface expression of CD25, CD44, CD71, CD98 and increased surface expression of CD62L.

75. The IDH2 inhibitor for use according to any one of embodiments 43 to 75, wherein the memory phenotype comprises an increased basal oxygen consumption, maximal respiratory capacity and/or space respiratory capacity compared to a control.

76. The IDH2 inhibitor for use according to embodiment 75, wherein the basal oxygen consumption is about 1.3-fold increased compared to a control.

77. The IDH2 inhibitor for use according to embodiments 75 or 76, wherein the maximal respiratory capacity is about 1.8-fold increased compared to a control.

78. The IDH2 inhibitor for use according to any one of embodiments 75 to 77, wherein the spare respiratory capacity is about 2.2-fold increased compared to a control.

79. The IDH2 inhibitor for use according to any one of embodiments 43 to 78, wherein the T-cells and/or B-cells are cultured in vitro or ex vivo for immunotherapy.

80. The IDH2 inhibitor for use according to any one of embodiments 43 to 79, wherein the T-cells and/or B-cells are expanded during culture.

81. The IDH2 inhibitor for use according to any one of embodiments 42 to 80, wherein the immunotherapy comprises adoptive cell transfer.

82. A composition comprising an IDH2 inhibitor for use according to any one of embodiments 42 to 81.

83. The composition for use according to embodiment 82, characterized in that it comprises an additional anti-cancer drug.

84. The composition for use according to embodiment 83, wherein the additional anti-cancer drug is a checkpoint inhibitor.

85. A population of T-cells and/or B-cells obtained by the method of any one of embodiments 1 to 41, in particular comprising a higher proportion of T-cells and/or B-cells with a memory phenotype.

86. The population of T-cells and/or B-cells of embodiment 85, wherein the cells are human, non-human primate or mouse cells.

87. The population of T-cells and/or B-cells of embodiments 85 or 86, wherein the T-cells comprise cytotoxic T-cells.

88. The population of T-cells and/or B-cells of any one of embodiments 85 to 87, wherein a subset or most of the T-cells and/or B-cells is/are activated.

89. The population of T-cells and/or B-cells of any one of embodiments 85 to 88, wherein a subset or most of the T-cells and/or B-cells with a memory phenotype is/are activated.

90. The population of T-cells and/or B-cells of any one of embodiments 85 to 89, wherein the memory phenotype comprises expression of one or more memory marker(s).

91. The population of T-cells and/or B-cells of any one of embodiments 85 to 89, wherein the memory phenotype comprises expression of the memory marker(s) CD62L and/or TCF1.

92. The population of T-cells and/or B-cells of any one of embodiments 85 to 91, wherein the memory phenotype comprises surface expression of the memory marker CD62L.

93. The population of T-cells and/or B-cells of any one of embodiments 85 to 92, wherein the memory phenotype comprises surface expression of the memory marker(s) CD27 and/or CD127.

94. The population of T-cells and/or B-cells of any one of embodiments 85 to 93, wherein the memory phenotype comprises expression of the memory marker(s) CCR7, CD27 and/or CD28.

95. The population of T-cells and/or B-cells of any one of embodiments 85 to 94, wherein the memory phenotype comprises absence of detectable surface expression or low surface expression of the non-memory marker KLRG1.

96. The population of T-cells and/or B-cells of any one of embodiments 85 to 95, wherein the memory marker(s) is/are upregulated compared to a DMSO control.

97. The population of T-cells and/or B-cells of any one of embodiments 85 to 96, wherein the non-memory marker(s) is/are downregulated compared to a DMSO control.

98. The population of T-cells and/or B-cells of any one of embodiments 85 to 97, wherein the T-cells and/or B-cells express one or more activation marker(s).

99. The population of T-cells and/or B-cells of any one of embodiments 85 to 98, wherein the T-cells show surface expression of the activation marker(s) CD25, CD44, CD71 and/or CD98.

100. The population of T-cells and/or B-cells of any one of embodiments 85 to 99, wherein the T-cells show surface expression of CD25, CD44, CD71, CD98 and increased surface expression of CD62L.

101. The population of T-cells and/or B-cells of any one of embodiments 85 to 100, wherein the memory phenotype comprises an increased basal oxygen consumption, maximal respiratory capacity and/or spare respiratory capacity compared to a DMSO control.

102. The population of T-cells and/or B-cells of embodiment 101, wherein the basal oxygen consumption is about 1.3-fold increased compared to a DMSO control.

103. The population of T-cells and/or B-cells of embodiments 101 or 102, wherein the maximal respiratory capacity is about 1.8-fold increased compared to a DMSO control.

104. The population of T-cells and/or B-cells of any one of embodiments 101 to 103, wherein the spare respiratory capacity is about 2.2-fold increased compared to a DMSO control.

105. The population of T-cells and/or B-cells of any one of embodiments 87 to 104, which are mouse cells and wherein the proportion of cytotoxic T-cells showing surface expression of CD62L after about 7 days of culture is about 60 to 75%.

106. The population of T-cells and/or B-cells of any one of embodiments 87 to 105, which are mouse cells, and wherein the mean fluorescence activity of CD62L in cytotoxic T-cells is about 1.5-fold to 2-fold increased compared to a DMSO control.

107. The population of T-cells and/or B-cells of any one of embodiments 85 to 106 for use in immunotherapy.

108. The population of T-cells and/or B-cells for use according to embodiment 107, wherein the immunotherapy is a T-cell therapy.

109. The population of T-cells and/or B-cells for use according to embodiments 107 or 108, wherein the immunotherapy is a therapy to treat cancer.

110. The population of T-cells and/or B-cells for use according to embodiment 109, wherein the cancer is resistant to chemotherapy, targeted therapy and/or antibody-mediated immunotherapy.

111. The population of T-cells and/or B-cells for use according to embodiments 109 or 110, wherein the cancer comprises metastases.

112. The population of T-cells and/or B-cells for use according to any one of embodiments 107 or 108, wherein the immunotherapy is a therapy to treat a chronic viral infection.

113. The population of T-cells and/or B-cells for use according to any one of embodiments 107 or 108, wherein the immunotherapy is a therapy to treat an autoimmune disease.

The invention is also characterized by the following figures, figure legends and the following non-limiting examples.

### BRIEF DESCRIPTION OF THE FIGURES

FIG. 1: Experimental layout describing the in vitro culture. OT1 cells were cultured in the presence of IL-2, Ovalbumin peptide N4 and IDH2i or DMSO for 3 days. At day 3, IDH2 inhibitor and Ovalbumin peptide were washed away and cells were cultured in the presence of IL-2 and IL-7 for 4 additional days.

FIG. 2: Flow cytometry gating strategy for CD8+ T-cells obtained from in vitro culture. Cell populations expressing low and high levels of CD62L can be clearly separated.

FIG. 3: IDH2 inhibition promotes a memory phenotype in CD8+ T-cells as shown by flow cytometry experiments including an unstained control. Histograms show the relative expression of surface markers at day 3 and 7 on OT1 cells treated with DMSO or AG221. The “unstained control” was stained with a live/dead marker and anti-CD8 antibody, but not any other antibody. Cells were gated on single viable CD8 positive cells.

FIG. 4: IDH2 inhibition promotes a memory phenotype in CD8+ T-cells as shown by flow cytometry experiments including two IDH2 small molecule inhibitor treatments. Histograms show the relative expression of surface markers at days 3 and 7 on OT1 cells treated with DMSO (full line), AG221 (dashed line) and AGI6780 (dotted line). Cells were gated on single viable CD8 positive cells.

FIG. 5: Quantification of the surface expression of the memory marker CD62L. (A) Percentage of cells expressing high or low levels of CD62L out of the total CD8+ T cell population. (B and C) Mean fluorescence intensity (MFI) of CD62L in the CD8+ T cells (B) or CD62L-high population (C), expressed as fold change compared to DMSO. Flow cytometry measurements were performed at day 7 of in vitro culture (also see FIG. 4). Cells were gated on single viable CD8 positive cells, and in (C) further on high CD62L expression. Data represents mean +/− SEM. *p<0.05 compared to DMSO.

FIG. 6: Quantification of the mRNA expression of the memory marker CD62L. Bar graphs representing Sell (CD62L) expression fold changes compared to DMSO for IDH2i (AG221) treated OT1 cells at day 3 and day 7 as measured by RT Q-PCR.

FIG. 7: Extracellular metabolic flux analyses to characterize the metabolic state. Oxygen consumption rate (OCR) at day 7 of activated OT1 T-cells treated with DMSO or IDH2i (AG221) as measured with a XF-96 Extracellular Flux Analyzer (Seahorse Bioscience). Oligomycin, a complex V inhibitor, blocks mitochondrial ATP synthase. FCCP (fluoro-carbonyl cyanide phenylhydrazone) uncouples ATP synthesis from oxygen consumption by the electron transport chain. Etomoxir inhibits CPT1alpha, and thus fatty acid oxidation. Rotenone and antimycin A inhibit the electron transport chain.

FIG. 8: IDH2 inhibition promotes a memory phenotype in CD8+ T-cells as revealed by the absolute quantification of extracellular metabolic flux analyses. (A) Oxygen consumption rate (OCR) at baseline. (B) Extracellular acidification rate (ECAR) at baseline. (C) Maximal respiratory capacity (MRC). (D) Ratio of OCR and ECAR measurements. Measurements were done at day 7 of in vitro culture of DMSO or IDH2i (AG221) treated cells with a XF96 Extracellular Flux Analyzer (Seahorse Bioscience). *p<0.05, ***p<0.001 by unpaired t test. Mean±SEM is shown.

FIG. 9: IDH2 inhibition promotes a memory phenotype in CD8+ T-cells as revealed by the relative quantification of extracellular metabolic flux analyses. (A) Fold change in basal oxygen consumption (OCR), (B) maximal respiration and (C) spare respiratory capacity of AG221-treated T-cells versus DMSO-treated T-cells. Data represents mean +/− SEM. *p<0.05 compared to DMSO.

FIG. 10: Experimental layout scheme of the in vivo tumor model. 10⁵ melanoma B16-OVA cells were subcutaneously injected into CD45.2+ mice 7 days before adoptive cell transfer (ACT) of 10⁵ OT1 cells previously treated in vitro with either DMSO or IDH2i (AG221. Blood samples were collected at day 14 post tumor engraftment.

FIG. 11: CD8+ T-cells treated with IDH2 maintain a memory phenotype upon adoptive cell transfer in vivo. (A) Fraction of transferred cells (CD8+, CD45.1+) out of CD8+ cells. (B) Fraction of MPEC (KLRG1^{low}, CD127+) out of transferred cells. (C) Fraction of SLEC (KLRG1^{high}, CD127−) out of transferred cells. (D) Fraction of T_{CM} (CD44+, CD62L+) out of transferred cells. Measurements were performed with cells obtained from the blood of mice 7 days post ACT (day 14 of the entire protocol). The mice have either received DMSO or IDH2i (AG221) treated CD8+ T-cells. MPEC: memory precursor effector cells; SLEC: short-lived effector cells; T_{CM}: central memory T cells. *p<0.05 by unpaired t test or one-way ANOVA. Mean±SEM is shown.

FIG. 12: IDH2 treatment during in vitro culture enhances the anti-tumor activity of adoptively transferred CD8+ T-cells in vivo. Tumor growth of B16-OVA tumor-bearing mice adoptively transferred with 10⁵ OT1 cells treated in vitro with DMSO or two structurally different IDH2i: AG221 (square) and AGI6780 (triangle). At the time of ACT, mice also received a vaccination with CpG-OVA. An additional control group received only a PBS injection (open circle). *p<0.05 by unpaired t test or one-way ANOVA. Mean±SEM is shown.

FIG. 13: Western blot analysis on mouse OT1 T-cells at day 7 that were activated with the ovalbumine peptide SIINFEKL and cultured with DMSO or IDH2 inhibitor (AG221) from d0-d3, showing the trimethylation of H3K4 (H3K4-3Me) and acetylation of H3K27 (H3K27-Ac).

FIG. 14: Histogram showing CD62L expression measured by flow cytometry in mouse T cells cultured for 7 days in the presence of the IDH2 inhibitor AG221 (dashed line) as compared to a DMSO control (full line).

FIG. 15: Generation of human CD8+ T-cells with a memory phenotype by in vitro culture with an IDH2 inhibitor. (A) Schematic of activation, IDH2 treatment (AG211) and restimulation of human umbilical cord blood mononuclear cells (CBMC). (B) Phenotypic analysis of human bulk CBMC at day 11 of in vitro culture and activation measured by flow cytometry. Left: histogram showing CD62L expression at day 11 in human CBMCs cultured with AG221 (dashed line) or DMSO (full line), measured by flow cytometry. Right: percentage of CD62L+ cells of CD8+ T-cells at day 11 upon treatment with AKTi (MK2206), IDH2i (AG221) or DMSO control. *p<0.05, compared to DMSO Graphs show mean±SEM. (C-E) The percentage of CD8+ T-cells that express either IFNγ (C), TNF (D) or IL2 (E) in response to a restimulation by PMA and Ionomycin, measured by flow cytometry. Data represents mean±standard error of mean. *p<0.05 compared to DMSO.

FIG. 16: Phenotypic analysis of human bulk PBMC at day 11 post in vitro activation. (A,B) Quantification of flow cytometry analysis at day 11 post-activation showing the percentage of CD62L+ CD8 T cells in the CD3/CD8 double positive T cell population (A) or in all living cells (B) from a culture of PBMCs treated with AKTi (MK2206), IDH2i (AG221) or DMSO control. (C,D) Quantification of flow cytometry analysis at day 11 post-activation showing the percentage of CD62L+ CD4 T cells in the CD3-positive/CD8-negative T cell population (C) or in all living cells (D). (E,F) Quantification of flow cytometry analysis at day 11 post-activation showing the percentage of CD8 T cells (E, identified as CD3/CD8 double positive) and CD4 T cells (F, identified as CD3-positive/CD8-negative) in all living cells of a culture of PBMCs treated with AKTi (MK2206), IDH2i (AG221) or DMSO control. *p<0.05, compared to DMSO Graphs show mean±SEM.

FIG. 17: Phenotypic analysis of mouse CD4 T cell activation upon AG221 treatment. (A) Schematic representation of mouse CD4 T cell activation and treatment. (B and C) Histogram representing flow cytometry data on CD62L expression in CD4 T cells upon treatment with DMSO or IDH2 inhibitor (AG221) (B), and the quantification thereoff (C).

### EXAMPLES

Methods and materials are described herein for use in the present disclosure; other, suitable methods and materials known in the art can also be used. The materials, methods, and examples are illustrative only and not intended to be limiting.

Example 1. The OT1/ovalbumin system to study the functionality of CD8+ T-cells. Mice were maintained in the animal facility of the University of Lausanne (UNIL). OT1 mice were bred on site and C57BL/6 (B6) mice were obtained from ENVIGO. The mice were not exposed to ovalbumin. Splenocytes comprising CD8+ T-cells were isolated from OT1 mice which contain transgenic inserts for mouse T-cell receptor genes Tcra-V2 and Tcrb-V5. To isolate splenocytes, whole spleens from OT1 mice were collected under sterile conditions. The spleen was smashed on a 70 μm cell strainer and single cells were collected by centrifugation. Red blood cells were lysed by resuspending the pellet in 2 ml Red Blood Cell Lysis Buffer (Qiagen) and incubated for 5 minutes at room temperature. The splenocytes comprised naïve CD8+ T-cells and antigen-presenting cells. The transgenic T cell receptor was designed to recognize ovalbumin (ova) residues 257-264 in the context of the MHC class I molecule H2Kb. The OT1 system was used to allow in vitro selection and activation of CD8+ T-cells that are directed against cells (artificially) expressing ovalbumin, for example cancer cells. This system thus allowed the inventors to compare the functional properties, in particular the anti-cancer activity, of CD8+ T-cells treated with different compounds during in vitro culture. A B16-Ova melanoma tumor cell line, previously generated in the laboratory of the inventors, was used to generate tumors in mice. The tumors functioned as targets of cultured OT1 CD8+ T-cells which allowed measurement of their activity. All experiments were performed in accordance with Swiss federal regulations and procedures approved by veterinary authority of the Canton de Vaud.

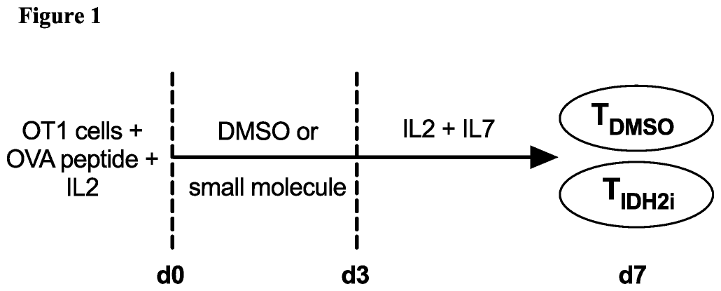

Example 2. In vitro culture of splenocytes comprising CD8+ T-cells. OT1 splenocytes (as described in Example 1) were cultured for 3 days at a concentration of 10⁶ cells per mL in RPMI medium (Gibco 61870-01) supplemented with 10% FCS, (Gibco 10270-106), 1% Penicillin/Streptomycin (P/S; Gibco 15070-063), 0.1% 13-mercaptoethanol, 1% HEPES (Gibco 15630-080), 1× Non-essential amino acids (Gibco 11140-035), 1% L-glutamine (Gibco 25030-081), 1 mM Sodium Pyruvate (Gibco 11360-039), 100 U/m1 hIL-2 (Glaxo-IMB) and 1 μg/ml ovalbumin N4 peptide (SIINFEKL). To evaluate the effects of IDH2 inhibition on T-cell differentiation, 5 μM of a small molecule IDH2 inhibitor (AG221 or AGI6780) or a DMSO solvent control was added during this 3-day culture period. At day 3, a fraction of the splenocytes was collected and the remaining cells were cultured for 4 additional days with 100 U/ml hIL-2 and 10 ng/ml hIL-7 (Peprotech 200-07) supplementation after washing out the inhibitors and the ovalbumine N4 peptide (FIG. 1). At day 3 and day 7, the activation and differentiation state of CD8+ T-cells was determined by gene expression analysis, flow cytometry and extracellular metabolic flux analyses (see Example 3).

Example 3. Determination of the T-cell activation and differentiation status. The expression of genes which mark different T-cell activation and differentiation states was measured using quantitative real time RT-PCR (see Example 4) and flow cytometry (see Examples 4 and 6). Since T-cells switch during differentiation from a rather lipid-based metabolism to increased aerobic glycolysis, oxygen consumption rates were determined by extracellular flux analyses to further characterize the differentiation state (see Example 4).

a) Gene expression analysis by quantitative real time RT-PCR RNA was isolated with the RNeasy mini kit (Qiagen) and cDNA was generated by reverse transcription (Applied Biosystems). Real-time RT-PCR was performed using primers against Sell (FW primer: 5′ atc tgc caa gtg ctc cag tg 3′, RV primer: 5′ gag ggg gtt gta gtc cag tg 3′). Gene expression was calculated relative to Δ2-microglobulin (FW primer: 5′ aga ctg ata cat acg cct gca g 3′, RV primer: 5′ gca ggt tca aat gaa tct tca g 3′).

b) Flow Cytometry

Cells were incubated in a live/dead stain (Fixable aqua dead cell stain kit, Invitrogen) followed by an incubation with different antibody panels.

Antibody panel to determine the differentiation and activation state of in vitro cultured T-cells (see Examples 2 and 4): anti-CD8-PE-texas-Red, anti-CD62L-FITC, anti-CD127-PE, anti-CD27-PerCP-Cy5.5, anti-CD71-PE-Cy7, anti-CD44-APC-Cy7, anti-CD98-APC, anti-CD25-Pacific blue (BD Pharmingen and eBioscience, San Diego, Calif., USA). All those antigens are surface molecules. Cells were gated on living single CD8+ cells before analysis of activation and memory marker expression. For the unstained control, cells were stained with the live/dead stain and anti-CD8-PE-texas-Red.

Antibody panel to determine the frequency and differentiation and activation state of adoptively transferred T-cells in the blood (see Example 6): CD8-PE-texas-Red, CD45.1-Pacific blue, CD45.2-FITC, CD127-PE, KLRG1-PE-Cy7, CD62L-APC, CD44-APC-Cy7, and CD27-PerCP-Cy5.5.

Cells were acquired on LSR-II flow cytometers from the flow cytometry facility of UNIL and data were analyzed with FlowJo™ 10 software. Antibodies were purchased from BD Phamingen (San Diego, Calif., USA), eBioscience (San Diego, Calif., USA, and Biolegend (San Diego, Calif., USA).

c) Extracellular Flux Analyses

Real-time analysis of CD8⁺ T cell oxygen consumption rate (OCR) was measured with an XF-96 Extracellular Flux Analyzer (Seahorse Bioscience) at day 7 of in vitro culture (FIG. 7). Measurements were performed while successively adding different drugs to the culture without any washing steps in between. Specifically, the measurements were performed under basal conditions, after addition of 1.25 μM oligomycin, a complex V inhibitor that blocks mitochondrial ATP synthase, after addition of 1 μM protonophore FCCP (fluoro-carbonyl cyanide phenylhydrazone) to uncouple ATP synthesis from oxygen consumption by the electron transport chain, after addition of 50 μM etomoxir to inhibit CPT1alpha, and thus fatty acid oxidation, and after addition of 100 nM rotenone and 1 μM antimycin A to inhibit the electron transport chain.

The basal OCR is the OCR measured in the absence of drugs. Addition of oligomycin allows to calculate the ATP-linked respiration by subtracting the oligomycin OCR from basal OCR. FCCP enables the electron transport chain (ETC) to reach its maximal rate, allowing to determine the cellular maximal respiratory capacity. Finally, rotenone and antimycin A inhibit the ETC which indicates the non-mitochondrial respiration. The spare respiratory capacity (SRC) is defined as the difference between basal OCR and maximal OCR obtained after FCCP addition.

In addition, the extracellular acidification rate (ECAR), which is an indicator for T-cell activation, was measured using the same device under basal conditions. This allowed further calculating the OCR/ECAR ratio as indicator of basal mitochondrial activity.

d) Statistical Analyses

Statistical analyses were performed in GraphPad Prism 7 software using different statistical tests indicated for each experiment. Results are shown in mean±SEM and P<0.05 was considered statistically significant.

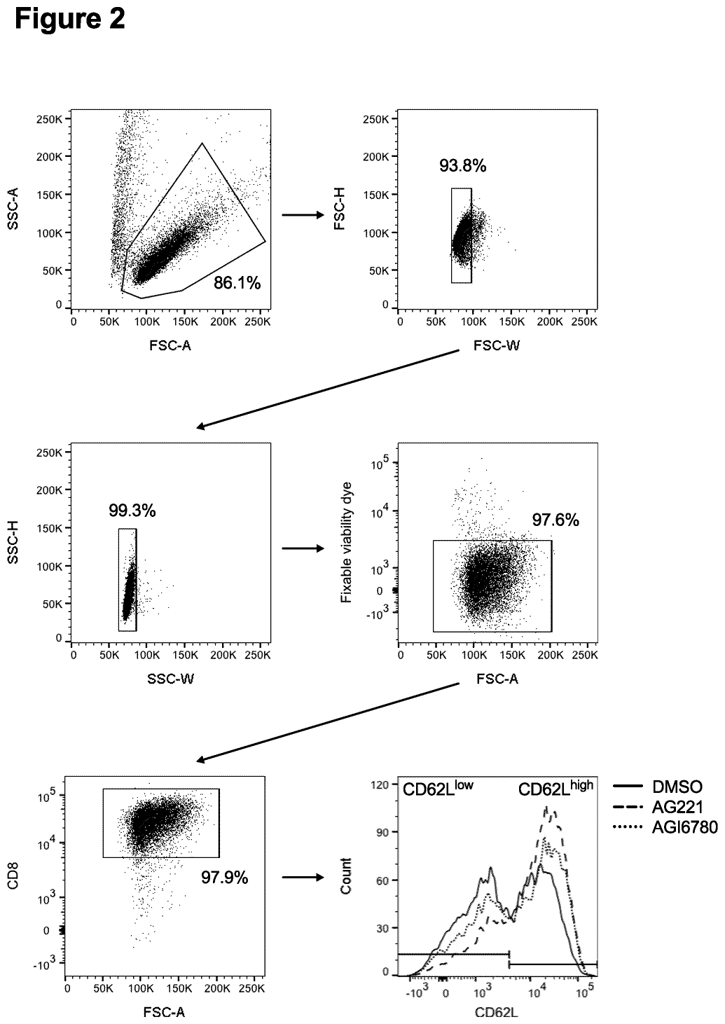

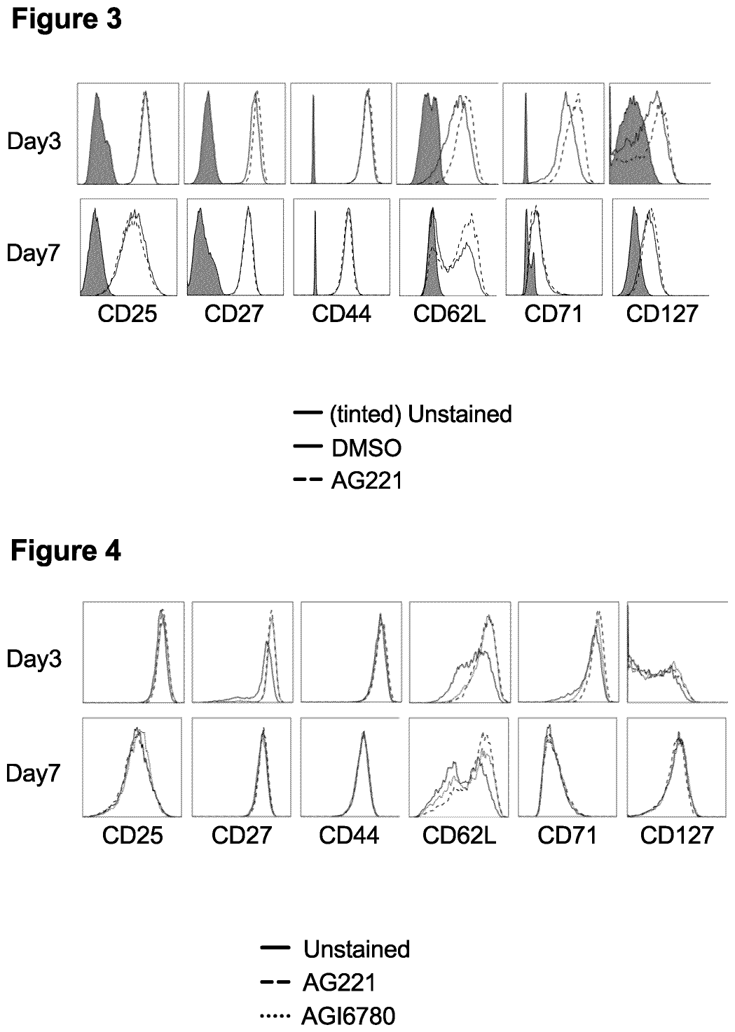

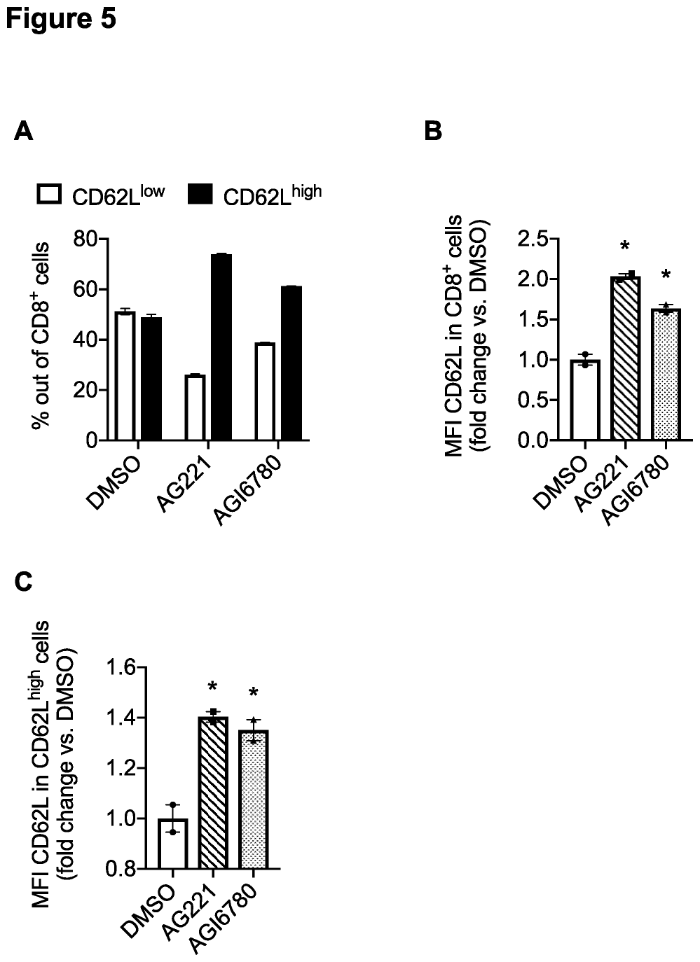

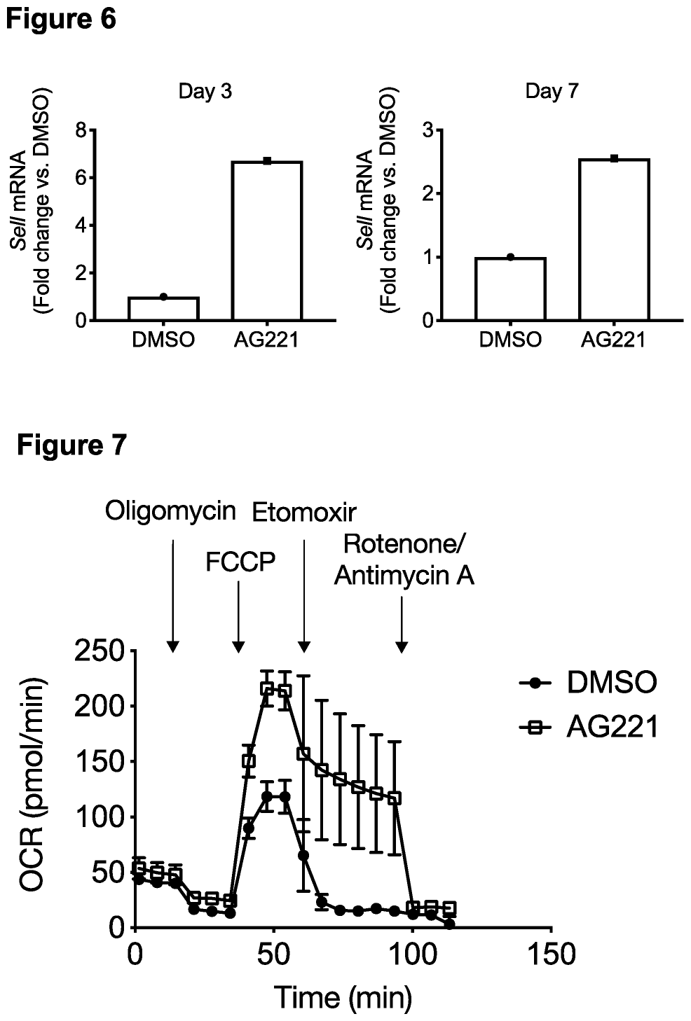

Example 4. IDH2 inhibition during CD8+ T cell activation increased memory characteristics in vitro. Surface activation marker (CD25, CD44, CD71, CD98) and surface memory marker (CD27, CD62L, CD127) expression was analyzed by flow cytometry on day 3 and 7 (FIGS. 2 to 5; also see Example 3). CD62L expression was increased in cells treated with an IDH2 inhibitor compared to DMSO, while the other markers were generally unchanged (FIGS. 3 to 5). The fact that AG221 and AGI6780 gave the same results suggests that the upregulation of CD62L is likely due to the specific inhibition of IDH2 (FIGS. 4 and 5). Furthermore, Sell (CD62L) mRNA expression (see Example 3) was increased 3-fold at day 3 and 7-fold at day 7 in cells treated with AG221 (FIG. 6). Thus, the increase in the memory marker CD62L upon AG221 in vitro treatment occurs, at least partly, at the transcriptional level.

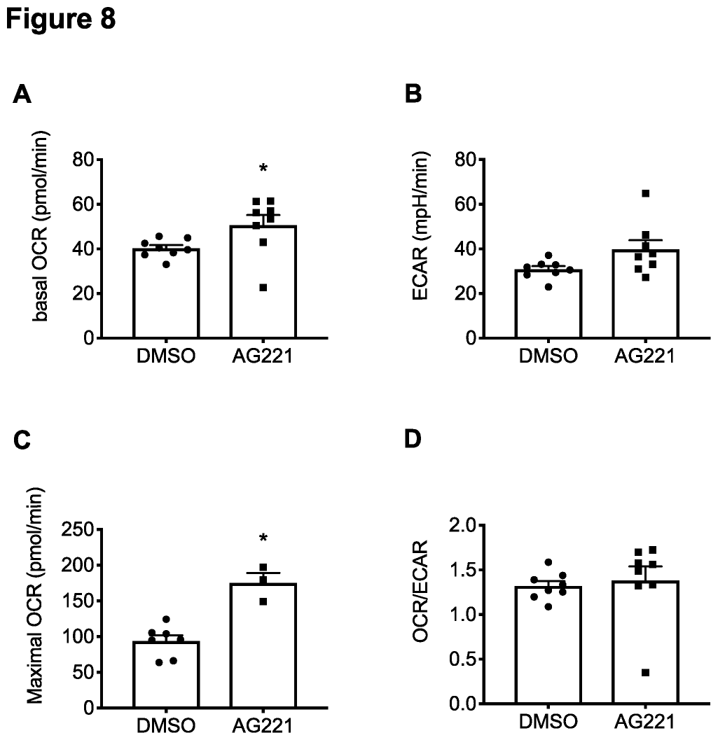

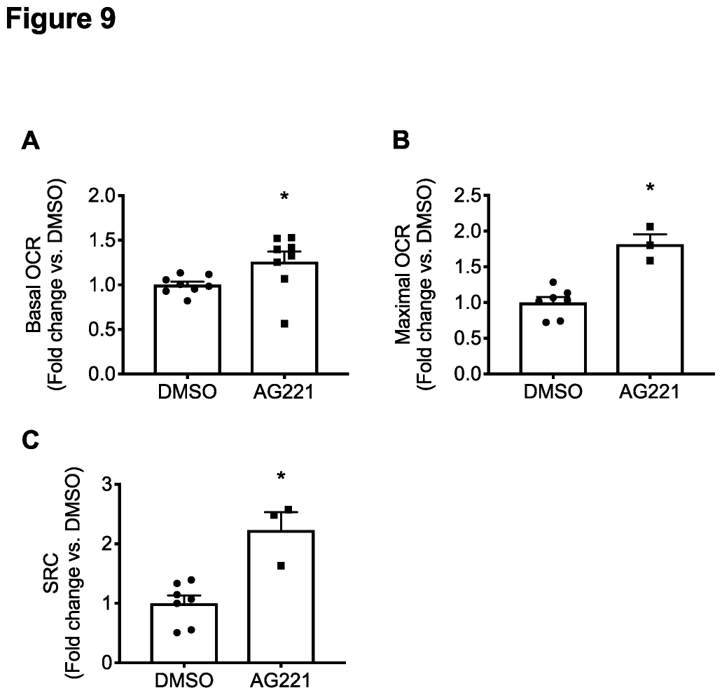

Moreover, extracellular metabolic flux analyses (see Example 3) at day 7 revealed that cells treated with AG221 had a significantly increased basal oxygen consumption rate (OCR), maximal respiratory capacity and spare respiratory capacity (SRC) compared to the control group treated with DMSO, a feature associated to memory CD8⁺ T cells (FIGS. 7 to 9). However, the extracellular acidification rates (ECAR), and the OCR/ECAR ratio were unchanged in AG221 treated cells compared to control group DMSO (FIGS. 8B, D). Together, these results indicate that transient IDH2 inhibition during in vitro T-cell activation enhanced the generation of memory-like CD8⁺ T cells. Yet, although transient IDH2 inhibition during in vitro T-cell activation is sufficient for enhancing the generation of memory-like CD8⁺ T-cells, the IDH2 inhibition does not have to be necessarily transient but IDH2 can be inhibited also during the entire time of culture to achieve such an effect, as demonstrated in Examples 9 and 10. In fact, contacting the T-cells with an IDH2 inhibitor for a longer time may enhance the generation of memory-like T cells even further.

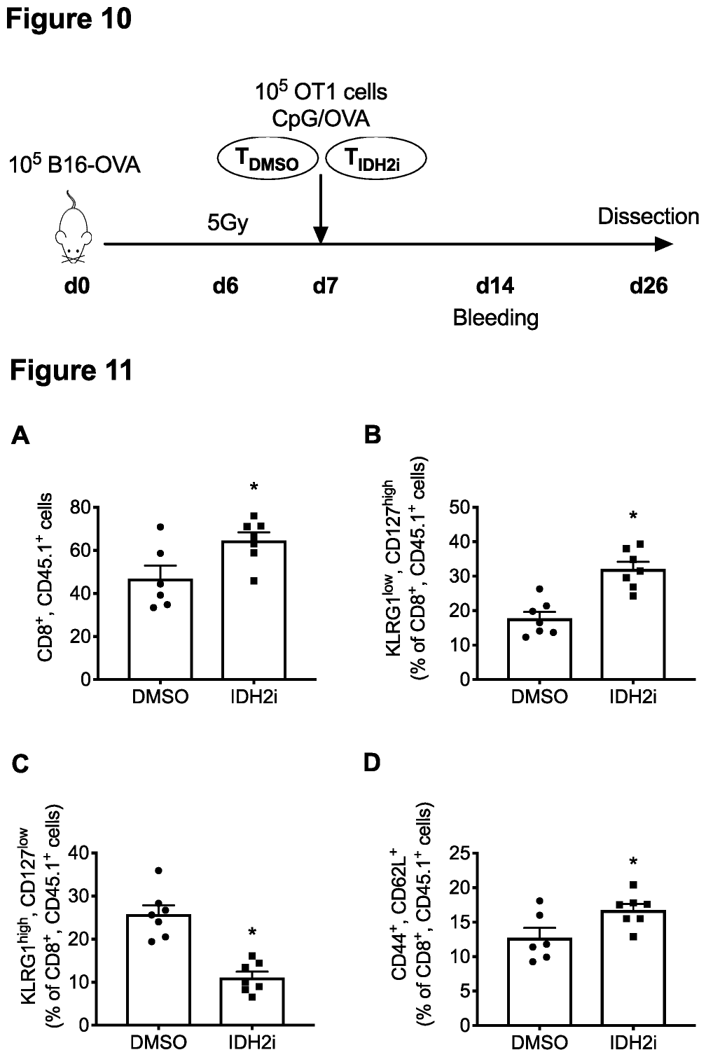

Example 5. In vivo tumor model to characterize the phenotype and efficacy of cultured CD8+ T-cells upon adoptive cell transfer. An in vivo tumor model was used to determine the efficacy of in vitro cultured CD8+ T-cells treated with an IDH2 inhibitor in eliminating cancer cells (FIG. 10). B16-OVA melanoma cells (see Example 1) were cultured in DMEM (GIBCO) with 10% FCS and 1% P/S before their subcutaneous injection into the mouse flank. Each mouse received 100′000 cells in a volume of 200 μl of PBS. 6 days after B16-OVA cell injection, tumors were measured, mice were randomized and lymphodepleted by irradiation (5 Gray). 7 days after B16-Ova cell injection, mice were adoptively transferred with activated CD45.1⁺ OT-1 splenocytes. The CD45.1⁺ OT-1 splenocytes had been cultured in vitro for 7 days as described in Example 2, wherein the IDH2 inhibitor was AG221, collected and purified on a Ficoll gradient to separate dead and live splenocytes. Live splenocytes, almost exclusively consisting of CD8+ T-cells (about 98%), were counted with Trypan blue stain 0.4%. 100′000 live splenocytes were transferred into CD45.2⁺ host mice by tail vein injection.

Following the adoptive cell transfer (ACT), mice received a vaccination of CpG (50 μg/mouse) and N4 Ova peptide (10 μg/mouse) diluted in PBS to obtain a total volume of 100 μl/mouse, injected subcutaneously at the tail base to further stimulate the transferred cells. An additional no-ACT control group received only a PBS injection (neither cells, CpG nor peptide). Tumors were measured every 2 days with a digital caliper and the tumor volume was calculated according to the formula: V=π×[d2×D]/6, wherein d is the smallest tumor axis and D is the largest tumor axis.

Example 6. Maintenance of the CD8+ T-cell memory phenotype in vivo. Blood samples were collected at day 14 and day 18 post tumor engraftment (see Example 5). At day 14 (day 7 post ACT), a significantly higher frequency of transferred T-cells was detected in mice when the transferred cells had been treated with the IDH2 inhibitor AG221 during in vitro culture (see Example 2) before ACT (FIG. 11A). Furthermore, the proportions of memory precursor effector cells (MPECs) and central memory T cells (T_{CM}) within the transferred CD8+ T-cells population at day 14 were significantly higher upon treatment with an IDH2 inhibitor compared to the DMSO control (FIGS. 11B, D), and the proportion of short-lived effector cells (SLECs) was significantly lower (FIG. 11C). Transferred CD8+ T-cells were CD8⁺ and CD45.1⁺. Among those, MPECs were KLRG1^{low} and CD127⁺, SLECs were KLRG1^{high} and CD127⁻, and T_{CM} were CD44⁺ and CD62L⁺.

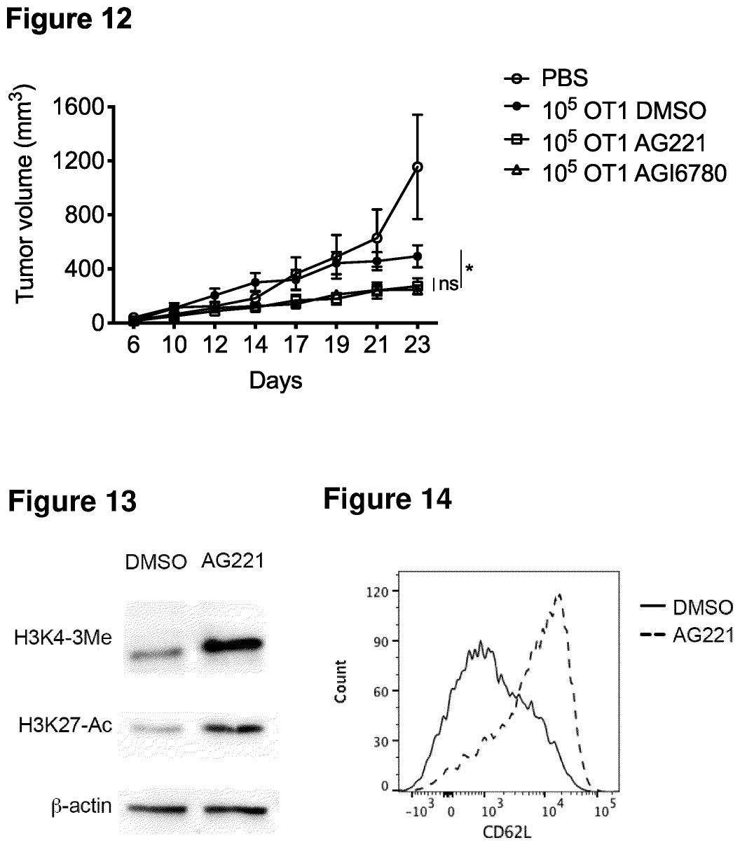

Example 7. Enhanced anti-tumor activity of adoptively transferred T-cells by IDH2 treatment. The tumor burden, in particular the tumor growth, was significantly reduced in the group of mice that was adoptively transferred with AG221 treated T-cells (see Example 5), indicating that these cells acquired, in addition to their memory phenotype (see Examples 4 and 6), an enhanced anti-tumor activity (FIG. 12).

Example 8. IDH2 treatment induces chromatin changes. T-cells treated for only 3 days with the IDH2 inhibitor (from d0-d3) still showed a strongly increased CD62L expression by d7 (see Example 4) that is even maintained upon further in vivo transfer (see Example 6). It was thus hypothesized that IDH2 inhibition induces certain epigenetic modifications allowing a sustained increased gene expression even long after the inhibitor has been removed. Therefore, the inventors analyzed by Western blot the acetylation and methylation status of histone residues that have been involved in memory CD8 T cell formation. Additionally, an ATAC-seq (Assay for Transposase-Accessible Chromatin using sequencing) was performed to quantify and identify the genes that are associated with open chromatin in DMSO versus AG221-treated T cells.

Methods: OT1 splenocytes were activated and cultured with 5 μM AG221 or DMSO as described in Example 2. After 3 days, the cells were washed and the IDH2 inhibitor was washed out as well. The cells were then cultured for an additional 4 days in the absence of AG221. At day 7, the cells were collected and washed once with PBS. 50′000 cells were collected for ATAC-seq while the remaining cell pellet was lysed in extraction buffer (20 mM Tris HCl, 150 mM NaCl, 1% Triton X-100, 10% glycerol, 5 mM EDTA) supplemented with Complete Mini protease inhibitor (Roche) for wester blotting.

For ATAC-seq cells were transposed with the Illumina Tagment DNA Enzyme and Buffer Small Kit according to the protocol described by Buenrostro et al. (Curr Protoc Mol Biol. 2015 Jan 5;109:21.29.1-21.29.9.). Fragments were sequenced on an Illumina NextSeq 500 instrument.

For western blotting, an equal amount of protein (as measure by the BCA protein assay kit, Thermo Scientific) was size-separated on a 12.5% polyacrylamide gel and transferred to a 0.2 μm nitrocellulose membrane (Bio-Rad). The following antibodies were used: β-Actin (AC-74) from Sigma Aldrich, H3K4-trimethyl (C42D8) and H3K27-acetyl (D5E4) from Cell Signaling, and appropriate HRP-labelled secondary antibodies (Santa Cruz). The signal was visualized with ECLTM Select Western Blotting Detection Reagent from Sigma Aldrich or SuperSignal West Femto Maximum Sensitivity Substrate from Thermo Scientific and acquired by the Fusion FX from Vilber.

Results and discussion: ATAC-seq revealed 1633 more accessible regions in the DNA of AG221-treated T-cells as compared to DMSO-treated T-cells. This indicates that the chromatin in IDH2-treated T-cells is in a more open conformation. Moreover, the inventors observed by western blot an increased trimethylation of lysine residue 4 and acetylation of lysine residue 27 of histone H3 (FIG. 13), which are posttranslational histone modifications know to be associated with open chromatin. This suggests that the open chromatin configuration may be achieved by posttranslational modifications on histone residues, allowing the de-condensation of the chromatin.

In summary, this data suggests that IDH2 inhibition induces posttranslational modifications on histones, thereby making several regions in the DNA more accessible. Several of these regions are located near memory-associated genes, facilitating their transcription and thus memory T cell differentiation.

Furthermore, it is known that IDH2 is an enzyme involved in the TCA cycle in the mitochondria, and that many metabolites of the TCA cycle are important co-factors or inhibitors of enzymes that are involved in epigenetic processes (e.g. histone de-acetylation/acetylation or de-methylation/methylation). It is thus contemplated that IDH2 inhibition induces the posttranslational histone modification through alteration of TCA metabolite abundances.

Example 9. In vitro culture of splenocytes comprising CD8+ T-cells with the continuous presence of an IDH2 inhibitor. The above Examples demonstrate that treating T-cells only from day 0 to day 3 with the IDH2 inhibitor AG221 is sufficient to induce a stable memory phenotype which is characterized, e.g., by a long-lasting upregulation of the memory marker CD62L as measured on day 7. Yet, the inventors sought to optimize and maximize memory induction and thus tested treatment of the T-cells with the IDH2 inhibitor AG211 over the entire course of the culture for 7 days.

Methods: Mouse OT1 splenocytes were obtained as described in Example 1, counted and seeded at 1×10⁶ cells/ml in a 48 well plate in RPMI (completed with 10% fetal bovine serum (FBS), 1% L-glutamine, 1% Penicillin/streptomycin, 1 mM sodium pyruvate, non-essential amino acids (GIBCO), 10 mM HEPES and 50 μM (3-mercaptoethanol), in the presence of 1 μg/ml ovalbumine-derived SIINFEKL peptide and 100 IU/ml recombinant human IL-2, and 5 μM AG221 or DMSO, and incubated in a humidified incubator at 37° C. for 72 h. At 72 h, cells were washed twice with medium, split in 4, and cultured for another 96 h in the presence of 100 U/ml rhIL-2 and 10 ng/ml rhIL-7, with 5 μM AG221 or DMSO. At day 7 of culture, cell viability and surface marker expression (CD62L) was analyzed by flow cytometry.

Results and discussion: FIG. 14 shows a robust induction of CD62L expression in nearly 100% of the T cells treated with the IDH2 inhibitor. This demonstrates that the time of treatment with IDH2 inhibition can be extended over the course of the entire culture period, which enhances CD62L expression even further.

Example 10. Generation of improved human memory T-cells through IDH2 inhibition. Due to the high clinical relevance, the inventors sought to verify the findings obtained in mouse T-cells illustrated in the above Examples with human T-cells. Specifically, to mimic the experimental conditions that were successful by using naïve mouse OT1 CD8 T-cells, the IDH2 inhibitor was tested on naïve human CD8+ T-cells from umbilical cord blood which are promising for being routinely used for therapy, similarly as hematopoietic stem cells from cord blood.

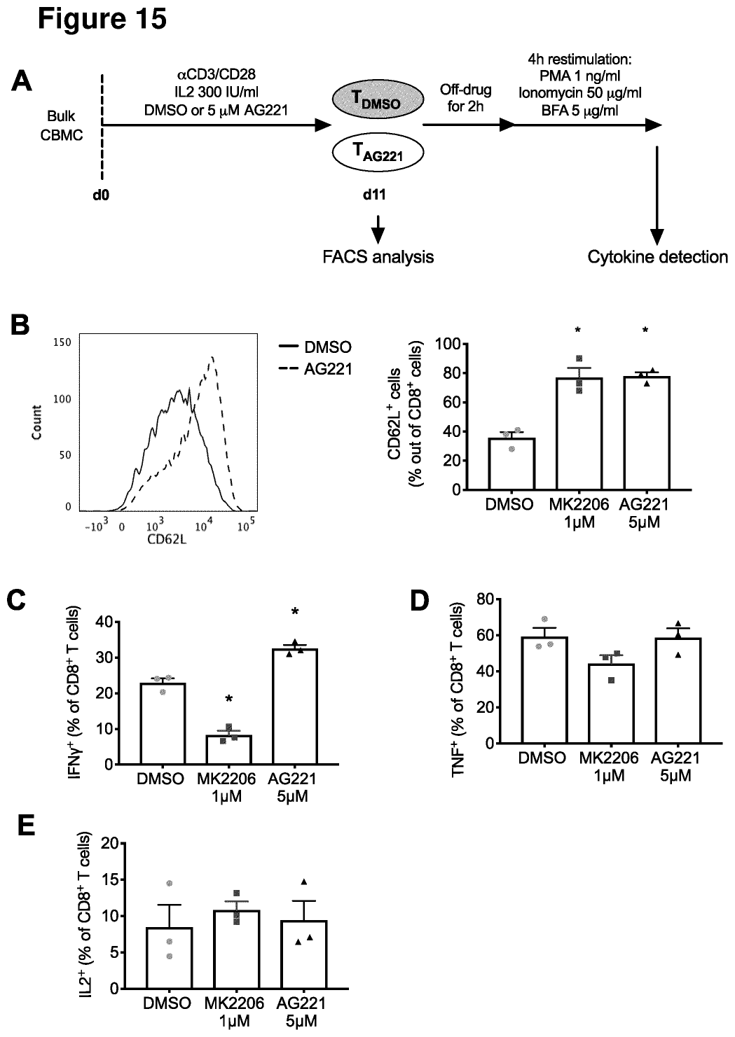

Methods: Mononuclear Cells were isolated from fresh umbilical cord blood on a Percoll Gradient (CBMCs). 10⁴ CBMC's were seeded per well in a round bottom 96-well plate in RPMI with 10% human serum, and activated with (magnetic) αCD3/CD28 at a 1:2 cell:bead ratio and 300 U/ml rhIL-2, in the presence of 5 μM AG221 or DMSO, or 1 μM MK2206 (AKT inhibitor). After 11 days of culture, CD62L expression was analyzed by flow cytometry on a fraction of the cells (FIG. 15A). The other cells were washed 2 times and placed in a magnet to remove the inhibitors and the αCD3/CD28 beads. The cells were then rested for 4 hours before restimulating them for 4 hours with 1 ng/ml Phorbol 12-Myristate 13-Acetate (PMA, a PKC activator) and 50 μg/ml Ionomycin (a Ca²⁺ ionophore, activating NF-KB and NFAT) in the presence of 50 μ/ml golgi inhibitor (Brefeldin A, BFA), allowing for the measurement of intracellular accumulation of different cytokines by flow cytometry.

Results and discussion: Flow cytometry analysis on d11 showed a strong upregulation of CD62L expression in AG221-treated human CD8+ T-cells (FIG. 15B). This suggests that the mechanism of memory induction upon AG221 treatment is conserved between mice and human. In particular, about 75% of the CD8+ cells in the culture treated with the IDH2 inhibitor AG221 or the AKT inhibitor MK2206 expressed CD62L compared to about 35% of the CD8+ cells in the control culture (DMSO without the inhibitors).

One of the main characteristics of memory T-cells is their ability to react with an increased amplitude to a reencounter of the antigen. Therefore, the inventors tested the ability of day11-treated human T cells to express the effector cytokines IFNγ and TNF and the proliferation-inducing cytokine IL-2. The inventors further included human T-cells that were treated with a small molecule inhibitor of AKT (MK2206) in their assay, since this was shown to induce CD62L expression in human T cells as well (Klebanoff et al., JCI Insight. 2017 Dec 7;2(23):e95103). First the small molecule inhibitors were washed out and the T cells were rested for 4 hours before they were restimulated with PMA and ionomycin which allows maximal and global non-TCR specific T-cell activation. It was surprisingly observed that AKT inhibition is detrimental for the expression of the effector cytokine IFNγ. AG221 on the other hand induced a significant upregulation of IFNγ (FIG. 15C). TNF expression was unaffected by inhibitor treatment (FIG. 15D) and the production of the proliferation-inducing cytokine IL-2 was unaltered upon inhibitor treatment (FIG. 15E). It is thus concluded that IDH2 inhibition results in the generation of a T-cell product that is able to respond to restimulation by increased effector cytokine production, and which is thus superior compared to AKT-inhibitor-treated cells.

Example 11. CD62L expression in whole culture and in CD4 T-cells

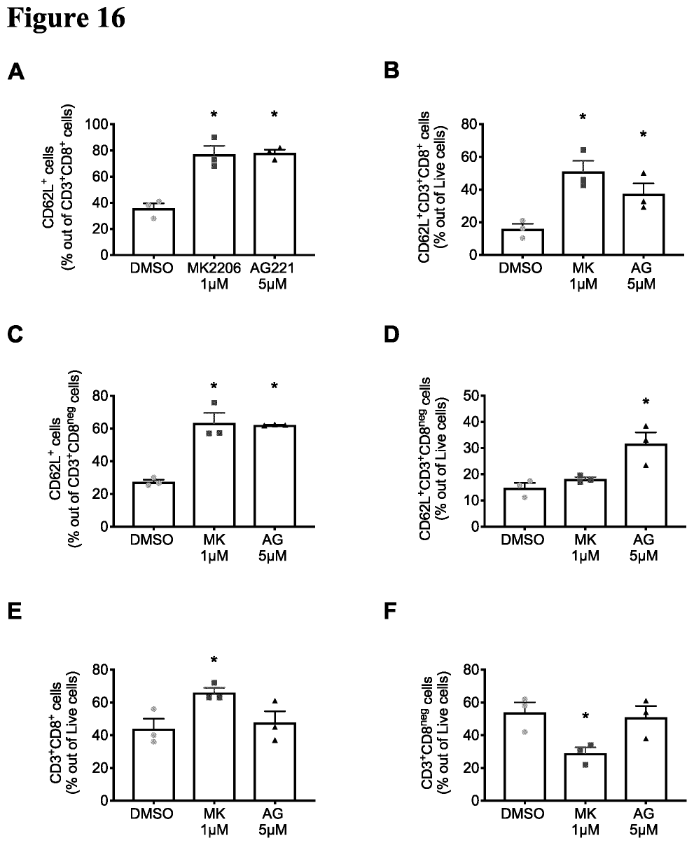

The previously obtained data has undergone additional analyses, in order to obtain more insight in CD62L expression in the whole culture as well as in CD4 T cells. In the human figure A and B, we now clearly made the distinction between CD62L expression as percentage out of CD8 T cells (FIG. 16A) or out of all living cells in the culture (FIG. 16 B).

As mentioned before, about 98% of the cells in culture on day 11 are CD3-positive. Since CD3 is exclusively expressed on CD4 and CD8 T cells, we can deduct that all CD3-positive, CD8-negative must be CD4 T cells. When analyzing the CD3-pos/CD8-neg population we observed that CD62L expression is induced upon AG221 treatment, both when expressed as percentage out of CD4 T cells (FIG. 16 C) and as percentage out of total living cells (FIG. 16 D). In figure D, we observed that AKT inhibition failed to increase the percentage of CD62L+ CD4 T cells out of all living cells in culture. This can be explained by our observation that AKT inhibition favors CD8 T cell differentiation over CD4 T cell differentiation. This ratio remains unchanged upon AG221-treatment, as compared to DMSO control (FIG. 16 E and F).

Example 12. IDH inhibition promotes memory marker expression in CD4 T-cells

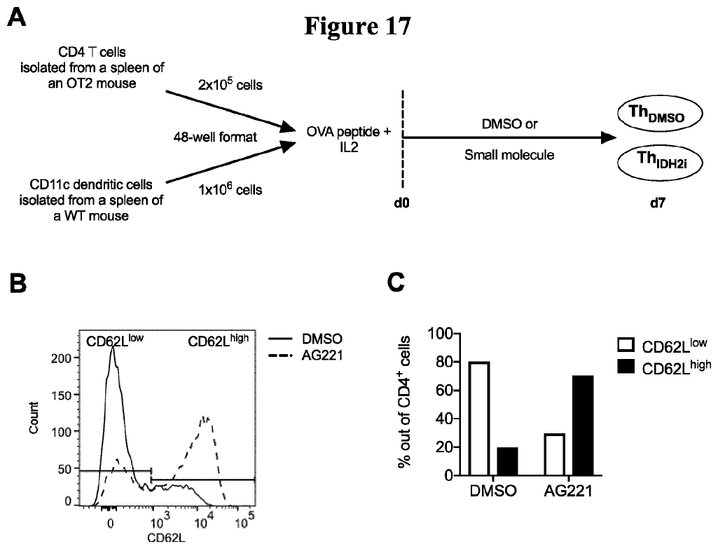

A new experiment has been performed on mouse T cells in order to further strengthen our claim that IDH2 inhibition also promotes memory marker expression in CD4 T cells. As indicated in the experimental scheme (FIG. 17A), we activated CD4 T cells from OT2 mice by co-culture with dendritic cells. OT2 mice are transgenic for an αβTCR recognizing specifically a chicken ovalbumine peptide 323-339 associated with the mouse MHC class II molecule I-A^{b}. We activated OT2 CD4 T cells by co-culture with dendritic cells loaded with 1 μg/l of the ovalbumine peptide in the presence of 100 IU/ml recombinant human IL2 in the presence of 5 μM AG221 or DMSO control. After 4 days, cells were collected, washed and split, and brought back in culture with 100 IU/ml rhIL2 and 5 μM AG221 or DMSO control. At day 7, CD62L expression was determined by flow cytometry and showed a strong increase in the CD62L-positive population upon IDH2i treatment (FIG. 17 B and C).

Measurement of IDH2 inhibitory activity:

AG-221 was prepared as 10 mM stock in dimethyl sulfoxide (DMSO) and diluted to 50+ final concentration in DMSO. IDH2 enzyme activity in converting isocitrate to αKG was measured in a continuous assay directly coupling NADPH production to conversion of resazurin to the fluorescent molecule resorufin by diaphorase. Resorufin was measured via fluorescence (λex=544 nm, γem=590 nm).

IDH2 homodimer enzyme was diluted to 0.1 μg/ml in 40 μl Assay Buffer (150 mM NaCl, 50 mM K2HPO4 pH 7.0, 10 mM MgCl2, 10% glycerol, 0.03% BSA) containing 1 μl of 50× compound dilution series, 44 μM NADP+ cofactor, and 2.5 μM β-mercaptoethanol. mixture was incubated at 25° C. for 16 hours. The reaction was then initiated with the addition of 10 μl of a substrate mix containing 0.2 mM isocitrate, 60 μg/ml diaphorase, and 200 μM resazurin in Assay Buffer. The reactions were run at 25° C. for 30 minutes, stopped by the addition of 25 μl of 6% SDS solution and read on a SpectraMax 384-well plate reader as described above.

1. In an in vitro cell culture method of culturing T-cells, the improvement for which comprises contacting said T-cells with an IDH2 inhibitor.

2. (canceled)

3. A method for generating and/or maintaining T-cells with a memory phenotype comprising the steps of:
(a) culturing T-cells in vitro and
(b) adding an IDH2 inhibitor to the culture of a)step (a).

4. The method according to claim 3, which further comprises the step of obtaining from the culture a cell population comprising T-cells with a memory phenotype.

5. (canceled)

6. (canceled)

7. The method according to claim 3, wherein the T-cells comprise CD8+ T-cells.

8. The method according to claim 3, wherein the T-cells are human autologous cells.

9. (canceled)

10. (canceled)

11. The method according to claim 3, wherein said culturing step comprises an activation phase and an expansion phase and said T-cells are contacted with the IDH2 inhibitor during at least a portion of the activation phase.

12. (canceled)

13. (canceled)

14. The method according to claim 11 wherein the T-cells are activated by contacting them with an antigenic peptide, optionally in the presence of antigen-presenting cells.

15. The method according to claim 11, wherein the T-cells are activated by contacting them with anti-CD3 and anti-CD28 antibodies.

16. The method according to claim 3, wherein the T-cells are further contacted with IL-2.

17. The method according to claim 3, wherein the culturing of the T-cells includes a medium comprising the (i) IDH2 inhibitor, (ii) IL-2, and (iii) anti-CD3 and anti-CD28 antibodies and/or an antigenic peptide.

18. The method according to claim 3, wherein the culturing of the T-cells comprises a first medium comprising the (i) IDH2 inhibitor, (ii) IL-2, and (iii) anti-CD3 and anti-CD28 antibodies and/or an antigenic peptide, and then in a second medium comprising IL-2 and IL-7.

19. The method according to claim 3, wherein the IDH2 inhibitor comprises at least one small molecule.

20. The method according to claim 19, wherein the at least one small molecule is selected from 2-methyl-1-[[4-[6-(trifluoromethyl)pyridin-2-yl]-6-[[2-(trifluoromethyl)pyridin-4-yl]amino]-1,3,5-triazin-2-yl]amino]propan-2-ol (AG221), 1-[5-(cyclopropylsulfamoyl)-2-thiophen-3-ylphenyl]-3-[3-(trifluoromethyl)phenyl]urea (AGI6780), and combinations thereof.

21. The method according to claim 20, wherein the concentration of AG221 and/or AGI6780 is 1, 2, 3, 4 or 5 micromolar.

22. A cell population comprising T-cells with a memory phenotype obtained by the method according to claim 3.

23. A cell population, wherein at least 90% of the cells are T-cells and at least 30%, of the cells in the population are T-cells with a memory phenotype.

24. The cell population according to claim 23, wherein at least 70% of the cells in the population are T-cells with a memory phenotype.

25-32. (canceled)

33. The cell population according to claim 23, wherein
(a) at least 90% of the cells in the cell population are human T-cells, and at least 35% of the cells in the cell population are human CD8+ T-cells that express CD62L.

4-37. (canceled)

38. The method according to claim 23, wherein the memory phenotype comprises expression of at least one memory marker selected from the group consisting of: CD62L, TCF1, CD27, CD127, CCR7 and CD28.

39-45. (canceled)

46. A method of immunotherapy, which comprises administering the cell population according to claim 22 to a patient.

47-50. (canceled)

51. The cell population according to claim 23, wherein the T-cells comprise a heterologous antigen receptor selected from a T-cell receptor (TCR) or a chimeric antigen receptor (CAR).

52. (canceled)

53. (canceled)
